(19) United States
(12) Patent Application Publication
(10) Pub. No.: US 2006/0084861 A1
(43) Pub. Date: Apr. 20, 2006

(54) Magnet and coil configurations for MRI probes

(75) Inventors: Aharon Blank, Kiryat-Ono (IL); Gadi Lewkonya, Neve-Mivtach (IL); Yuval Zur, Haifa (IL); Hanna Friedman, Givat-Zeev (IL); Gil Tidhar, Modiin (IL)

Correspondence Address: WOLF, BLOCK, SCHORR & SOLIS-COHEN LLP, 250 PARK AVENUE, NEW YORK, NY, 10177, US

(73) Assignee: TopSpin Medical (Isreal) Ltd., Lod (IL)

(21) Appl. No.: 10/968,853

(22) Filed: Oct. 18, 2004

(51) Int. Cl. A61B 5/05; A61B 6/00; A61M 25/00

(52) U.S. Cl. 600/423; 600/427; 600/433

(57) ABSTRACT

A probe, with a longitudinal axis, for use in an NMR system, the probe comprising: (a) a plurality of static magnetic field sources which create a static magnetic field that is non-axisymmetric about the longitudinal axis, in a region outside the probe; and (b) at least one antenna, compromising one or more antennas together capable of creating a time-varying magnetic field which is capable of exciting nuclei in a sub-region of the region, and capable of receiving NMR signals from said excited nuclei and generating NMR electrical signals therefrom; wherein the plurality of magnetic field sources comprise adjacent static magnetic field sources that are magnetized in directions that differ by more than 10 degrees and less than 170 degrees.

### RELATED APPLICATIONS

This application is related to a patent application titled “Expanding MRI Probe,” attorney's docket number 334/03529, filed on even date, at the US Patent and Trademark Office, the disclosure of which is incorporated herein by reference.

### FIELD OF THE INVENTION

The field of the invention is nuclear magnetic resonance probes.

### BACKGROUND OF THE INVENTION

Problems with Conventional MRI

Conventional MRI (magnetic resonance imaging) systems suffer from a number of limitations. They require highly homogeneous magnetic fields, which, for imaging a large volume such as the human body, generally means large and expensive equipment that is not very mobile. The distance between the imaged volume and the RF antenna means that a rather long acquisition time is needed to obtain a reasonable signal to noise ratio at high resolution. Because most patients cannot tolerate being inside the narrow bore of a large magnet for more than a few minutes, the images have limited resolution, typically about 1 millimeter. While “open” MRI magnets exist, with less claustrophobic bores, they have lower magnetic fields which further reduces the signal to noise ratio, so these systems also typically have resolution no better than 1 mm. Conventional MRI systems are thus unable to resolve plaque in the thin walls of arteries, even though MRI, in contrast to other imaging techniques, is very good at distinguishing between different types of soft tissues.

“Inside Out” MRI

These limitations of conventional MRI have led to the development of newer MRI techniques, in which smaller volumes can be imaged with higher resolution, and/or with less expensive and more portable equipment. Often, the region being imaged is outside the magnet and the RF coil, rather than being surrounded by the magnet and RF coil, as in conventional MRI.

One problem in “inside out” medical MRI, is that a high magnetic field gradient is generally produced outside the magnet. For a fixed small RF bandwidth typical of those used in conventional MRI, higher field gradients make the resonant region narrower, with fewer nuclei to produce a signal, and allow the nuclei to diffuse away from the resonant region quickly, further reducing the signal. If the bandwidth is made wider, then the noise increases. In either case, the signal to noise ratio (SNR) is small. This problem can also be at least partly overcome by using appropriate magnet configurations. One such configuration is described by Jackson, U.S. Pat. No. 4,350,955, the disclosure of which is incorporated herein by reference. Jackson describes two magnets, arranged along the z-axis with a gap between them, and magnetized in opposite directions parallel to the z-axis. This configuration produces a ring surrounding the probe where the magnetic field intensity has a saddle point, at which the magnetic field gradient is relatively small. Other magnet configurations which produce a saddle point in the magnetic field intensity outside the probes are described by Clow, U.S. Pat. No. 4,629,986, by Masi, U.S. Pat. No. 4,717,876 (including both axially and radially magnetized magnets in an axisymmetric probe), by Locatelli U.S. Pat. No. 5,610,522, and by Kleinberg et al, “Novel NMR Apparatus for Investigating an External Sample”, J. Magn. Res., 97, p. 466, 1992, the disclosures of which are incorporated herein by reference.

Medical MRI Receiver Probes

Most MRI probes used for medical applications are not self-contained probes with a magnet and RF transmitter, but only have an RF receiver, which is used inside the body to pick up signals from a small region of interest, while a conventional MRI magnet and RF transmitter are located outside the body to excite the region. Such probes are described by Atalar, U.S. Pat. No. 5,699,801, by Bradley, U.S. Pat. No. 5,050,607, by Kandarpa et al., J. Vasc. and Interventional Radiology, 4, pp. 419-427, 1993, and by H. H. Quick et al, Magnetic Resonance in Medicine 42:738-745, 1999, the disclosures of which are incorporated herein by reference. Sometimes the probe also produces a local field gradient, either using coils, described by Young, U.S. Pat. No. 5,303,707, or using pieces of soft magnetic material, described by Golan, U.S. Pat. No. 6,377,048, the disclosures of which are also incorporated herein by reference.

Self-Contained Medical MRI Probes

Pulyer, U.S. Pat. No. 5,572,132, the disclosure of which is incorporated herein by reference, describes a medical MRI probe which is self-contained, including a magnet, gradient coils, and RF transmitting and receiving coils. It has magnets (two magnets both magnetized in the same direction along the z-axis, with a gap between them) carefully shaped to produce a limited region of low magnetic field gradient outside the probe. The probe can be used for NMR spectroscopy, as well as for imaging.

Throughout this application, we will refer to the longitudinal direction, for example in a blood vessel, as the z direction, with the x and y directions perpendicular to the z direction and to each other.

Westphal et al, in U.S. Pat. No. 5,959,454, the disclosure of which is incorporated herein by reference, describes an MRI probe with an external imaging region on one side, for use outside the body to examine skin, for example.

Cho, U.S. Pat. No. 5,023,554, and Kikinis, U.S. Pat. No. 5,390,673, the disclosures of which are incorporated herein by reference, describe using a very inhomogeneous static magnetic field, and imaging slices that are far from flat, for medical MRI.

Crowley, U.S. Pat. No. 5,304,930, the disclosure of which is incorporated herein by reference, describes an MRI device located just outside the body, and used to image a region of the body. Prado et al, in U.S. Pat. No. 6,489,767, the disclosure of which is incorporated herein by reference, describes a palm-sized MRI probe with a planar imaging region on one side.

The NMR-MOUSE, a mobile NMR sensor, is described by Todica et al, J. Magn. Res. 164 (2003) 220-227, by Klein et al, J. Magn. Res. 164 (2003) 310-320, and by references therein. Anferova et al, “Construction of a NMR-MOUSE with Short Dead Time,” Concepts in Magnetic Resonance (Magnetic Resonance Engineering) 15(1), 15-25 (2002) describes ways of designing the coil and other components in an NMR-MOUSE which result in a dead time in the RF receiver amplifier of only 20 microseconds, after transmitting RF pulses through the same antenna. The RF frequency is about 20 MHz. This dead time is much shorter than the dead times in conventional MRI systems which use the same RF antenna for transmission and receiving, as described, for example, by Eiichi Fukushima and Stephen B. W. Roeder, in Experimental Pulse NMR: A Nuts and Bolts Approach, Perseus Publishing, 1986. The disclosures of all these articles and this book are incorporated herein by reference.

In U.S. Pat. No. 6,704,594, the disclosure of which is incorporated herein by reference, Blank et al describe a self-contained intravascular MRI probe. The probe uses two cylindrical magnets, arranged along the z-axis, magnetized in opposite directions perpendicular to the z-axis. This configuration, with an RF transmitting and receiving coil on one side of the probe, produces sector-shaped imaging slices on that side of the probe, with limited axial and radial extent.

### SUMMARY OF THE INVENTION

Non-axisymmetric Magnet Configurations

An aspect of some embodiments of the invention concerns self-contained MRI probes, for example intravascular probes, which have novel magnet and/or RF coil configurations that may result in improved properties compared to the prior art. Improved properties may include one or more of: higher static magnetic field in the imaging region for a given strength magnet; greater radial penetration of the static magnetic field and the RF field into the region surrounding the probe; a field of view with greater axial extent; and the ability to image regions in more than one azimuthal direction, and/or at one more than one axial position, simultaneously, without any need to rotate the probe or to move the probe axially.

In some of these novel configurations, there are a plurality of magnets arranged along the z-axis of the probe, configured so that the magnetic field is not axisymmetric, and adjacent magnets have directions of magnetization that differ by an angle that is substantially different from 0 degrees or 180 degrees. For example, the directions differ by an angle that is between 10 degrees and 170 degrees. Optionally, the directions differ by an angle that is between 20 degrees and 160 degrees, or between 40 degrees and 140 degrees.

For example, there are two magnets, the bottom one being magnetized at an angle of 45 degrees between the +z direction and the +x direction, and the top one being magnetized at an angle of 45 degrees between the +z direction and the −x direction. This configuration, in which the direction of magnetization differs by 90 degrees for the two magnets, produces a magnetic field just to the +x side of the probe that is greater than the magnetic field just to the −x side of the probe, and greater than the field would be just outside both sides of the probe if the two magnets were magnetized in opposite directions along the x-axis.

In another example, there are a plurality of magnets arranged axially along the probe. Each magnet is magnetized in a direction perpendicular to the z-axis, but in a different direction in the x-y plane that differs by less than 180 degrees from the direction of magnetization of the next magnet. For example, there are four magnets, magnetized respectively in the +x direction, the +y direction, the −x direction, and the −y direction. Each of these magnets has its own RF coil, and is used to obtain imaging data from a different azimuthal direction. Since all four magnets can produce data simultaneously, there is no need to take data first in one azimuthal direction, then rotate the probe, then take data in another azimuthal direction, etc., and a complete scan can be done more quickly. Even if the data from all the different magnets is not obtained simultaneously, but some or all of it is obtained sequentially, for example to avoid interference between magnets, this probe configuration still saves time because it is not necessary to rotate the probe. It also provides greater accuracy, because the difference in azimuthal direction between the different magnets is fixed by the structure of the probe, while in a probe which must be rotated, there may be an error in the angle of rotation.

Although the different magnets are located at different axial positions, this may not make much difference in certain applications, for example in examining arteries for plaque, because plaque tends to extend some distance along arteries at a same azimuthal location. Optionally, additional magnets are located further away axially, where the plaque is likely to look different, and the additional magnets are used to obtain data at different axial locations, simultaneously or sequentially, without the need to move the probe axially.

Optionally, in these probes, there is a structure which allows the probe to expand radially, for example inside a blood vessel, pressing each magnet, and/or its associated RF coil, against the wall of the blood vessel in a different azimuthal direction. Optionally, each magnet presses against the wall with its direction of magnetization normal to the wall at that point, maximizing the field intensity in the imaging region, for each position around the wall azimuthally. Alternatively, each magnet presses against the wall with its direction of magnetization parallel to the wall, and with an associated RF coil adjacent to the wall, producing an RF magnetic field normal to the wall.

Configurations with Longitudinally Magnetized Magnets

An aspect of some embodiments of the invention concerns self-contained MRI probes, for example intravascular probes, which have two magnets arranged along the z-axis, with direction of magnetization respectively in the +z and −z direction, and substantially no gap between the magnets. With no gap between the magnets, there is no saddle point in the magnetic field, surrounded by a region of low field gradient, within the field of view of the probe. Alternatively, there is a gap between at least some adjacent magnets, but the region of low field gradient is still outside the field of view.

Alternatively, there are three, four, or more magnets arranged along the z-axis, with direction of magnetization alternately in the +z and −z direction. These configurations, extended with a sufficiently large number of magnets magnetized alternately in the +z and −z directions, make it possible to produce a field of view which extends an arbitrary distance longitudinally. Optionally, there is a region of low field gradient, even within the field of view.

In some embodiments of the invention, various coil configurations are used, where optimal configurations make efficient use of the static magnetic field and RF magnetic field to obtain NMR data. Optimally, to make efficient use of the fields, the RF magnetic field should be close to perpendicular to the static magnetic field, and close to its maximum intensity, over most of the volume where the static magnetic field is comparable to its maximum intensity. Optionally, this volume of maximum or near maximum fields extends over a length that is a significant fraction of the radial dimension of the probe, radially out to a distance beyond the surface of the probe that is at least a significant fraction of the radius of the probe, and over a significant range of azimuthal angles. Efficient use of the static and RF magnetic fields increases the SNR, or reduces the RF heating of tissue for each bit of imaging data at a given SNR, and increases the field of view for a given probe size.

Other Magnet Configurations

An aspect of some embodiments of the invention concerns a magnet for a self-contained MRI probe. The magnet is magnetized in the x-direction, and has a circular cross-section but excluding one or two sub-volumes on their periphery. The sub-volumes may, for example, be volumes outside planes that are, for example, delineated by chords, parallel to the x-axis, on one or both sides of the circumference. In this case, the missing pieces of the magnet would contribute very little to the magnetic field strength just outside the magnet near the x-axis, which is the location where the field is greatest, and where the imaging region is located if the magnet is being used efficiently. Optionally, the volume that these missing sub-volumes took up is used for electronic circuitry, or for a balloon. Alternatively, the imaging region is located outside the magnet near the y-axis, but there is only one missing sub-volume, on the side of the magnet opposite the imaging region. In this case too, the missing sub-volume of the magnet would contribute relatively little to the magnetic field in the imaging region.

Optionally, the circular cross-section of the magnet is not a solid circle but a hollow circle, and, with the missing piece removed, the cross-section of the magnet is C-shaped. The hollow in the center is used, for example, to allow blood to flow through the probe, for a guide wire, and/or for cables which connect to the RF antenna.

An aspect of some embodiments of the invention concerns an MRI probe with a long cylindrical magnet (not necessarily a circular cylinder), and with an end cap at one or both ends. The end cap is optionally made of a high permeability material such as iron, and/or is sufficiently thick, and has high enough saturation flux density, to carry a substantial fraction of the flux of the magnet within one diameter of the end. The end cap may make the magnetic field around the magnet more uniform as a function of longitudinal position, possibly over most of the length of the magnet, and may make the field fall off more abruptly near the end of the magnet. This may make the imaging region, which may be limited by the contours of field strength, more uniform over the length of the magnet, potentially improving the signal to noise ratio. A more uniform imaging region may also provide more accurate radial voxel assignment for imagining the blood vessel wall, therefore making it possible to accurately estimate the distance of the plaque from the edge of the lumen which is an important parameter in evaluating its vulnerability. The magnet is magnetized substantially perpendicular to the axis of the cylinder.

An aspect of some embodiments of the invention concerns an MRI probe with a cylindrical permanent magnet and an RF coil, with the RF coil located in a shallow depression in the surface of the magnet, rather than located outside the outer diameter of the magnet. The parts of the magnet which extend to the same outer diameter as the RF coil, to the sides of the RF coil, are referred to herein as “ears.” Depending on the dimensions of the coil and the magnet, this configuration produces a higher static magnetic field in the imaging region of the probe, and hence higher resolution, higher signal to noise ratio, or shorter acquisition time, for the same permanent magnet material and the same outer envelope of the probe.

Reduced Eddy Currents

An aspect of an embodiment of the invention concerns an MRI probe with reduced eddy currents. The probe comprises an RF coil (or another kind of RF antenna or RF transmitting element) and a permanent magnet, with a design which reduces eddy currents induced in the magnet at the RF frequency when the RF coil transmits RF power. In an exemplary embodiment of the invention, there is a gap between the RF coil and the magnet. In an exemplary embodiment of the invention, there is a layer of a good conductor, such as copper, between the RF coil and the magnet, which shields the magnet, but not the imaging volume, from the RF fields generated by the RF coil. Although the conductor will also have eddy currents, they will generally dissipate less power than eddy currents in the lower conductivity magnet without shielding, since the skin depth is generally small. In an exemplary embodiment of the invention, the magnet is laminated, or has slots in it. Optionally, any gap between the RF coil and the magnet, or any layer of conductor between the RF coil and the magnet, is thick enough to significantly reduce eddy currents in the magnet. But optionally, the gap is not so thick as to significantly reduce the static magnet field in the imaging region by reducing the volume of the magnet for a given probe envelope, or by increasing the distance between the magnet and the imaging region. Optionally, the gap and/or the thickness of the conductor is optimized by minimizing a function that reflects both the adverse effect of eddy currents in the magnet, and the adverse effect of reducing the static magnetic field in the imaging region.

Non-imaging NMR with Self-contained Probes

Any of the MRI probes described above are also optionally used for non-imaging NMR. For example, instead of creating an image by obtaining NMR data from different longitudinal, azimuthal or radial positions relative to the probe, optionally the NMR data from all positions is lumped together, to obtain information about an average, possibly a weighted average, of the NMR characteristics of material in the field of view of the probe. This is done, for example, in order to increase the SNR of the signal, or to decrease the acquisition time for a given SNR, at the cost of losing information about the spatial distribution of the material. Losing information about the spatial distribution might not matter very much if it is expected that the material is distributed fairly broadly over the field of view of the probe. The NMR characteristics comprise, for example, the density of protons and/or other nuclei, T₁, T₂, the diffusion rate, and/or spectroscopic data. Such characteristics are optionally used, for example, to distinguish plaque from healthy tissue in the walls of blood vessels, even without imaging.

Such non-imaging NMR data may be obtained with any self-contained NMR or MRI probe, not just with the probes described above, including probes for which the static magnetic field has a saddle point around which the field is locally uniform. Although the presence of a locally uniform magnetic field region may make the probe particularly suitable for obtaining spectroscopic data, such a probe is also useful for obtaining data on density, T₁, T₂, and diffusion rate. An aspect of an embodiment of the invention concerns an NMR system using a self-contained NMR probe, whether the probe is suitable for imaging or not, which system is used to measure spatially averaged non-imaging NMR characteristics, other than spectroscopic data, and where there is a saddle point in the static magnetic field outside the probe.

RF Antenna with Short Dead Time

An aspect of some embodiments of the invention concerns an active protection circuit with very high bandwidth. The protection circuit isolates a sensitive low noise amplifies used to amplify weak received NMR signals, from an RF antenna, when the antenna is transmitting high power RF pulses. The same RF antenna can thus be used for both receiving and transmitting. The protection circuit uses active elements, such as toroid protectors, and optionally also uses passive elements such as a high pass filter and Schottky diodes. The circuit has a very high bandwidth and short ringing time, allowing the amplifier to have a very short dead time, as short as a few microseconds.

There is thus provided, in accordance with an exemplary embodiment of the invention, a probe, with a longitudinal axis, for use in an NMR system, the probe comprising:
(a) a plurality of static magnetic field sources which create a static magnetic field that is non-axisymmetric about the longitudinal axis, in a region outside the probe; and
(b) at least one antenna, compromising one or more antennas together capable of creating a time-varying magnetic field which is capable of exciting nuclei in a sub-region of the region, and capable of receiving NMR signals from said excited nuclei and generating NMR electrical signals therefrom;
   wherein the plurality of magnetic field sources comprise adjacent static magnetic field sources that are magnetized in directions that differ by more than 10 degrees and less than 170 degrees.

Optionally, said adjacent static magnetic field sources are displaced from each other along the longitudinal axis.

Optionally, adjacent static magnetic field sources are magnetized in directions that differ by more than 20 degrees and less than 160 degrees.

Optionally, adjacent static magnetic field sources are magnetized in directions that differ by more than 40 degrees and less than 140 degrees.

In an embodiment of the invention, the probe is adapted for inserting into a cavity in the body.

Optionally, the probe is adapted for inserting into a blood vessel.

Optionally, the probe is adapted for inserting into a blood vessel with inner diameter between 1.5 mm and 6 mm.

Optionally, the probe is adapted for inserting into a blood vessel with inner diameter between 2 mm and 4 mm.

In an embodiment of the invention, the static magnetic field sources comprise a first magnetic field source and a second magnetic field source, both with longitudinal components of magnetization having a same sign, and with transverse components of magnetization differing in direction by more than 90 degrees.

Optionally, there is a gap between the first and second magnetic field sources.

Optionally, the transverse components of magnetization differ in direction by more than 140 degrees.

Optionally, the transverse components of magnetization differ in direction by more than 160 degrees.

Optionally, the ratio of the magnitude of the transverse and longitudinal components of magnetization is greater than 0.5 and less than 2, for both the first and second magnetic field sources.

Optionally, the ratio is between 0.8 and 1.2, for both the first and second magnetic field sources.

In an embodiment of the invention, at least one of the at least one antennas extends over a range in the longitudinal direction that overlaps the longitudinal ranges of both the first and second magnetic field sources, and is located on one side of the longitudinal axis.

Optionally, the center of said antenna is located within 60 degrees of the location at which the longitudinal component of the static magnetic field is greatest, for that longitudinal position and distance from the longitudinal axis.

Optionally, the center of said antenna is located within 30 degrees azimuthally of said location.

In an embodiment of the invention, the first and second magnetic field sources extend radially to the surface of a smallest convex volume which includes both magnetic field sources, except for a slot carved into one or both of the first and second magnetic field source, and said antenna is located in one or both slots, entirely within said smallest convex volume.

Optionally, the smallest convex volume is cylindrical.

Optionally, the static magnetic field sources each have a component of magnetization transverse to the longitudinal axis that has a magnitude more than 2 times the magnitude of the longitudinal component of magnetization.

Optionally, the transverse component has a magnitude more than 5 times the magnitude of the longitudinal component.

Optionally, the transverse components of magnetization of adjacent static magnetic field sources differ in direction by more than 40 degrees and less than 140 degrees.

Optionally, the at least one antennas comprise an antenna associated with each of the static magnetic field sources.

Optionally, for each of said antennas, the static magnetic field in the extended sub-region is at least 80% produced by the static magnetic field source which that antenna is associated with.

Optionally, each sub-region has a limited range of azimuthal angles, and the azimuthal direction of the center of the range differs by more than 40 degrees and less than 140 degrees for at least two antennas associated with adjacent static magnetic field sources.

Optionally, the azimuthal direction of the center of the range for each of said antennas differs from the transverse component of the direction of magnetization (or the direction opposite to the direction of magnetization) of the static magnetic field source associated with that antenna by a same angle, to within ±20 degrees.

Optionally, the azimuthal direction of the center of the range for each of said antennas differs from the transverse component of the direction of magnetization (or the direction opposite to the direction of magnetization) of the static magnetic field source associated with that antenna by less than 20 degrees.

Alternatively, the azimuthal direction of the center of the range for each of said antennas differs from the transverse component of the direction of magnetization (or the direction opposite to the direction of magnetization) of the static magnetic field source associated with that antenna by between 70 and 110 degrees.

Optionally, the azimuthal direction of the center of the range for each of said antennas differs from the direction of the transverse component of the time-varying magnetic field produced by that antenna in the center of its sub-region, by less than 20 degrees.

Alternatively, the azimuthal direction of the center of the range for each of said antennas differs from the direction of the transverse component of the time-varying magnetic field produced by that antenna in the center of its sub-region, by between 70 and 110 degrees.

In an embodiment of the invention, the set of all azimuthal directions that are included within the range of any of said antennas does not have a gap greater than 90 degrees.

Optionally, the set of all azimuthal directions that are included within the range of any of said antennas does not have a gap greater than 45 degrees.

Optionally, the set of all azimuthal directions that are included within the range of any of said antennas covers more than 180 degrees.

Optionally, the set of all azimuthal directions that are included within the range of any of said antennas covers 360 degrees.

In an embodiment of the invention, the probe includes an expansion mechanism which, when it expands, moves at least two of the magnetic field sources, and its associated antenna, in different directions transverse to the longitudinal axis.

Optionally, the expansion mechanism moves each of the at least two static magnetic field sources in a direction that differs from the azimuthal direction of the center of the range for the antenna which that static magnetic field source is associated with, by a same angle, to within ±20 degrees.

Optionally, the expansion mechanism moves each of the at least two static magnetic field sources in a direction that differs from the azimuthal direction of the center of the range for the antenna which that static magnetic field source is associated with, by less than 20 degrees.

Optionally, the direction in which the expansion mechanism moves each of the at least two static magnetic field sources differs from the transverse component of the direction of magnetization (or the direction opposite to the direction of magnetization) of that static magnetic field source by a same angle, to within ±20 degrees.

In an embodiment of the invention, the probe is adapted to be inserted into a lumen of inner diameter greater than a minimum size, and when the imaging probe is inserted into a lumen of inner diameter twice the minimum size and the expansion mechanism is in its expanded state, the at least two static magnetic field sources and their associated antennas are close enough to the wall of the lumen so that at least part of the sub-region of each of their associated antennas is inside the wall.

Optionally, at least 40% of the NMR signal power received by said associated antennas originates from excited nuclei inside the wall.

Optionally, the parts of said sub-regions within the wall cover a set of azimuthal angles around the wall that does not have any gap greater than 90 degrees.

Optionally, the set of azimuthal angles around the wall does not have any gap greater than 45 degrees.

Optionally, the set of azimuthal angles around the wall covers more than 180 degrees.

Optionally, the set of azimuthal angles around the wall covers 360 degrees.

Optionally, the parts of said sub-regions within the wall cover said set of azimuthal angles within a longitudinal range of less than 15 mm.

In an embodiment of the invention, the time-varying magnetic field that the antenna associated with at least one of the static magnetic field sources creates is predominantly a dipole field outside the imaging probe.

Optionally, said antenna comprises two coils, adjacent to opposite sides of said static magnetic field source, which two coils run in phase with each other, and the time-varying magnetic field that said antenna creates in the center of the sub-region of said antenna is primarily transverse to the longitudinal axis.

Optionally, said antenna comprises a coil which wraps around said static magnetic field source longitudinally, and the time-varying magnetic field that said antenna creates in the center of the sub-region of said antenna is primarily transverse to the longitudinal axis.

Optionally, the dipole field has a dipole moment oriented at an angle greater than 45 degrees from the longitudinal axis.

Optionally, the static magnetic field that said static magnetic field source creates is predominantly a dipole field outside the imaging probe, and the dipole moment of the static magnetic field is oriented at an angle greater than 45 degrees from the dipole moment of the time-varying magnetic field.

Optionally, the dipole moment of the static magnetic field is oriented at an angle greater than 45 degrees to the longitudinal axis.

In an embodiment of the invention, the probe includes an expansion mechanism with a retracted state and an expanded state, which mechanism, when it expands, moves at least two of the static magnetic field sources in different directions transverse to the longitudinal axis.

In an embodiment of the invention, the probe is adapted to be inserted into a lumen of inner diameter greater than a minimum size, and, when the imaging probe is inserted into a lumen of inner diameter twice the minimum size and the expansion mechanism is in its expanded state, the probe presses against the wall of the lumen with sufficient force to stabilize the position of the probe sufficiently so that relative motion of the probe and the wall does not substantially affect the image quality.

Optionally, the probe is adapted to be inserted into an artery, and, when the lumen is an artery, the probe presses against the wall with no more than one atmosphere of pressure.

Optionally, the expansion mechanism comprises an expanding basket mechanism.

Alternatively or additionally, the expansion mechanism comprises an expanding helical mechanism.

Optionally, the expansion mechanism comprises a shape memory alloy.

Optionally, the expansion mechanism expands from the collapsed state to the expanded state when the temperature of the shape memory alloy is raised from below to above a shape memory transition temperature.

Alternatively or additionally, the shape memory alloy is in a superelastic state.

In an embodiment of the invention, the expansion mechanism comprises a distal end and a proximal end, and the expansion mechanism expands from the collapsed state to the expanded state when the distal end and the proximal end are brought closer together.

Alternatively or additionally, the expansion mechanism comprises a balloon, and the expansion mechanism expands from the collapsed state to the expanded state when the balloon is expanded.

Optionally, the plurality of static magnetic field sources comprises two static magnetic field sources.

Optionally, the plurality of static magnetic field sources comprises three static magnetic field sources.

Optionally, the plurality of static magnetic field sources comprises four static magnetic field sources.

Optionally, the plurality of static magnetic field sources comprises more than four static magnetic field sources.

In an embodiment of the invention, the sub-regions together have a longitudinal extent greater than 20% of the length of the probe in the longitudinal direction.

Optionally, the sub-regions together have a longitudinal extent greater than 50% of the length of the probe in the longitudinal direction.

Optionally, the sub-regions together have a longitudinal extent greater than 2 mm.

Optionally, the sub-regions together have a longitudinal extent greater than 5 mm.

Optionally, the sub-regions together have a longitudinal extent greater than 15 mm.

Optionally, the sub-regions together have a longitudinal extent greater than 30 mm.

In an embodiment of the invention, at least one of the static magnetic field sources is a permanent magnet element in the shape of a cylinder with a piece sliced off, the plane of the slice being within 20 degrees of parallel to the axis of the cylinder, the permanent magnet being magnetized in a direction substantially perpendicular to the axis of the cylinder and parallel to the plane of the slice.

There is further provided, in accordance with an exemplary embodiment of the invention, a probe with a longitudinal axis, for use in an NMR system, the probe comprising:
(a) a plurality of static magnetic field sources which together create a static magnetic field outside the probe that, in the absence of external magnetic field sources, has a magnitude which is a monotonic function of distance from the longitudinal axis, for any fixed values of longitudinal position and azimuthal angle; and
(b) at least one antenna, capable of creating a time-varying magnetic field which is capable of exciting nuclei in a sub-region of the region, and capable of receiving NMR signals from said excited nuclei and generating NMR electrical signals therefrom;
   wherein at least some of the static magnetic field sources are arranged in a row along the longitudinal axis, and adjacent sources are magnetized in opposite directions parallel to the longitudinal axis.

Optionally, the static magnetic field sources arranged in the row comprise three magnetic field sources.

There is further provided, in accordance with an exemplary embodiment of the invention, a probe with a longitudinal axis, for use in an NMR system, the probe comprising:
(a) at least three static magnetic field sources which create a static magnetic field in a region outside the probe; and
(b) at least one antenna, comprising one or more antennas together capable of creating a time-varying magnetic field which is capable of exciting nuclei in a sub-region of the region, and capable of receiving NMR signals from said excited nuclei and generating NMR electrical signals therefrom;
   wherein at least some of the static magnetic field sources are arranged in a row along the longitudinal axis, and adjacent elements are magnetized in opposite directions parallel to the longitudinal axis.

Optionally, the static magnetic field sources arranged in the row comprise four magnetic field sources.

Optionally, the at least one antenna comprises a plurality of coils, one for each static magnetic field source in the row, that is not located at an end of the row.

Optionally, each of the coils in the plurality of coils is located on a same side of the probe, adjacent to a different one of the static magnetic field sources in the row, that is not located at an end of the row.

Optionally, the at least one antenna comprises a coil.

Optionally, at least two of the static magnetic field sources in the row touch each other.

Alternatively or additionally, at least two of the adjacent static magnetic field sources in the row are separated by a gap.

Optionally, the gap at its narrowest point is smaller than 20% of the largest diameter of the probe at the gap.

Optionally, the plurality of static magnetic field sources comprise a plurality of permanent magnets.

There is further provided, in accordance with an exemplary embodiment of the invention, a probe for use in an NMR system, the probe comprising:
(a) a permanent magnet element in the shape of a cylinder with a piece sliced off, the plane of the slice being within 20 degrees of parallel to the axis of the cylinder, which magnet element creates a static magnetic field in a region outside the probe; and
(b) at least one antenna, comprising one or more antennas together capable of creating a time-varying magnetic field which is capable of exciting nuclei in a sub-region of the region, and capable of receiving NMR signals from said excited nuclei and generating NMR electrical signals therefrom;
   wherein the permanent magnet element is magnetized in a direction substantially perpendicular to the axis of the cylinder and parallel to the plane of the slice.

Optionally, the permanent magnet element is in the shape of a hollow right circular cylinder with the piece sliced off, and the slice extends into the hollow part of the cylinder, thereby making the permanent magnet element C-shaped.

Optionally, the probe is adapted to be inserted into a lumen, and includes a balloon which fits into the volume of the removed slice when the balloon is in a deflated state, and which holds the imaging probe against the wall of the lumen when the balloon is in an inflated state.

Optionally, the antenna is located on a different side of the cylinder than the slice.

Optionally, the cylinder is a right circular cylinder, the permanent magnet element has a slot carved into the side of the cylinder where the antenna is located, and the antenna is located in the slot, thereby confining the antenna substantially to the envelope of the cylinder.

Optionally, the permanent magnet element is in the shape of a cylinder with two pieces sliced off, the plane of each of the two slices being within 20 degrees of parallel to the axis of the cylinder.

Optionally, the two slices are within 20 degrees of parallel to each other. Optionally, the two slices are on different sides of the cylinder.

Optionally, the cylinder is a right circular cylinder.

Optionally, the probe includes an electrical component associated with the antenna, which component is located outside the surface of the slice, and within the cylinder.

In an embodiment of the invention, the plurality of static magnetic field sources comprise a permanent magnet, with substantially uniform cross-section transverse to the longitudinal axis, magnetized substantially uniformly in a direction substantially perpendicular to the longitudinal axis, and including at least one end cap, located at one end of the permanent magnet, sufficiently thick and permeable to make the magnetic field at a distance ⅔ of the magnet radius beyond the outer surface of the magnet vary by less than 10% longitudinally between the center of the magnet and a point ⅘ of the magnet radius away from said end of the magnet.

There is further provided, in accordance with an exemplary embodiment of the invention, a probe for use in an NMR system, the probe comprising:
a) a permanent magnet with a longitudinal axis, with substantially uniform cross-section transverse to the longitudinal axis, magnetized substantially uniformly in a direction substantially perpendicular to the longitudinal axis;
b) at least one antenna, comprising one or more antennas together capable of creating a time-varying magnetic field which is capable of exciting nuclei in a sub-region of the region, and capable of receiving NMR signals from said excited nuclei and generating NMR electrical signals therefrom; and
c) at least one end cap, located at one end of the permanent magnet, sufficiently thick and permeable to make the magnetic field around the permanent magnet substantially more uniform over most of the length of the permanent magnet, and to make the magnetic field around the permanent magnet fall off substantially more abruptly near said end of the permanent magnet, than if there were no end cap.

Optionally, the at least one end cap is sufficiently thick and permeable to make the magnetic field at a distance ⅔ of the magnet radius beyond the outer surface of the magnet vary by less than 10% longitudinally between the center of the magnet and a point ⅘ of the magnet radius away from said end of the magnet.

Optionally, the at least one end cap comprises two such end caps, located at each end of the permanent magnet.

Optionally, the at least one end cap has a thickness at least equal to one tenth of the diameter of the permanent magnet in the direction of magnetization.

Optionally, the time-varying magnetic field differs in direction from the static magnetic field by more than 60 degrees and less than 120 degrees, somewhere in the sub-region.

Optionally, at least one of the static magnetic field sources comprises a material with skin depth greater than the largest dimension of said static magnetic field source, at the proton nuclear resonance frequency at the maximum static magnet field in the region outside the probe.

Optionally, at least one of the static magnetic field sources comprises sintered material.

Optionally, at least one of the static magnetic field sources comprises ferrite.

Optionally, the probe is an imaging probe, and the NMR system is an MRI system.

Optionally, the one or more antennas comprise a single antenna capable of creating the time-varying magnetic field, and receiving the NMR signals and generating the NMR electrical signals.

Alternatively or additionally, the one or more antennas comprise:
(a) a transmitting antenna capable of creating the time-varying magnetic field; and
(b) a receiving antenna capable of receiving the NMR signals and generating the NMR electrical signals.

There is further provided, in accordance with an exemplary embodiment of the invention, an NMR system comprising a probe according to an embodiment of the invention, a power supply which transmits power to at least one of the antennas of the probe to create the time-varying magnetic field, and a data analyzer which reconstructs NMR characteristics of material in the sub-region from the NMR electrical signals generated by at least one of the antennas of the imaging probe.

Optionally, all of the at least one antennas that the power supply transmits power to are different from all of the at least one antennas that generate the NMR electrical signals from which the data analyzer reconstructs the NMR characteristics.

Alternatively, at least one of the at least one antennas both creates the time-varying magnetic field and generates the NMR electrical signals from which the data analyzer reconstructs the NMR characteristics.

Optionally, the NMR system is an MRI system, the probe is an imaging probe, and the data analyzer comprises an image reconstructor which reconstructs an image.

There is further provided, in accordance with an exemplary embodiment of the invention, an NMR system comprising:
a) a self-contained NMR probe with an RF antenna used for transmitting RF pulses and receiving NMR signals;
b) an amplifier for amplifying the NMR signals; and
c) an electric circuit, comprising active toroid protectors, which circuit isolates the amplifier from the RF antenna when the RF antenna is transmitting RF pulses, and connects the amplifier to the RF antenna when the RF antenna is receiving NMR signals.

There is further provided, in accordance with an exemplary embodiment of the invention, a non-imaging NMR system, comprising:
a) a probe, adapted for use inside the body, comprising a static magnetic field source which generates a static magnetic with at least one saddle point in a region outside the probe, and at least one antenna, comprising one or more antennas together capable of creating a time-varying magnetic field which is capable of exciting nuclei in a sub-region of the region, and capable of receiving NMR signals from said excited nuclei and generating NMR electrical signals therefrom;
b) a power supply which transmits power to at least one of the antennas of the probe to create the time-varying magnetic field; and
c) a data analyzer which reconstructs NMR characteristics, other than spectroscopic data, of material in the sub-region from the NMR electrical signals generated by at least one of the antennas of the imaging probe, but which data analyzer does not reconstruct images.

### BRIEF DESCRIPTION OF THE DRAWINGS

Exemplary embodiments of the invention are described in the following sections with reference to the drawings. The drawings are generally not to scale and the same or similar reference numbers are used for the same or related features on different drawings.

FIG. 1A shows a cross-section in the x-z plane of an MRI probe, according to an exemplary embodiment of the invention;

FIGS. 1B and 1C show cross-sections in the x-z plane of two different MRI or NMR probes, according to the prior art;

FIG. 1D shows a cross-section in the x-y plane of the MRI probe shown in FIG. 1A, at the bottom of the upper magnet;

FIG. 1E schematically shows an MRI system according to an exemplary embodiment of the invention, using the MRI probe shown in FIG. 1A;

FIG. 2A shows a perspective view of magnets in an MRI probe before the probe is deployed inside a blood vessel, according to an exemplary embodiment of the invention;

FIG. 2B shows a cross-sectional view, perpendicular to the longitudinal axis, of one of the magnets in FIG. 2A;

FIG. 3 shows a perspective view of an MRI probe deployed inside a blood vessel, utilizing the MRI probe according to the embodiment of FIG. 2;

FIG. 4 shows a perspective view of an MRI probe deployed inside a blood vessel, according to another exemplary embodiment of the invention;

FIG. 5 shows a cross-section in the x-z plane of an MRI probe, according to another exemplary embodiment of the invention;

FIG. 6 shows a cross-section in the x-z plane of an MRI probe, according to another exemplary embodiment of the invention;

FIG. 7A shows a cross-section perpendicular to the longitudinal axis, of an MRI probe, according to another exemplary embodiment of the invention;

FIG. 7B shows a perspective view of an MRI probe, according to another exemplary embodiment of the invention;

FIG. 8 shows a cross-section perpendicular to the longitudinal axis, of an MRI probe, according to another exemplary embodiment of the invention;

FIG. 9A is a perspective view, and FIG. 9B is a cross-sectional view perpendicular to the longitudinal axis, of an MRI probe or a sub-probe (for example one of the sub-probes in FIG. 2, 3, or 4) according to another exemplary embodiment of the invention;

FIG. 9C is a cross-sectional view perpendicular to the longitudinal axis, of the MRI probe or sub-probe in FIG. 9B, inside a blood vessel;

FIG. 10A is a perspective view, and FIG. 10B is a cross-sectional view perpendicular to the longitudinal axis, of an MRI probe or a sub-probe according to another exemplary embodiment of the invention;

FIG. 11 is a perspective view of an MRI probe or a sub-probe according to another exemplary embodiment of the invention;

FIG. 12A and FIG. 12B are plots of the contours of field strength in the y-z plane, at the plane of symmetry, for the MRI probes shown in FIGS. 10A and 11 respectively;

FIGS. 13A and 13B show two different perspective views of part of an MRI probe, according to the same embodiment shown in FIG. 1A;

FIG. 14 shows a circuit diagram of an electric circuit for the MRI probe partially shown in FIGS. 13A and 13B;

FIG. 15 is a perspective view of the MRI probe partially shown in FIGS. 13A and 13B, including also the layout of the rest of the electric circuit; and

FIG. 16 is a circuit diagram of an electric circuit used to isolate a low noise amplifier during RF transmission, according to an exemplary embodiment of the invention.

### DETAILED DESCRIPTION OF EXEMPLARY EMBODIMENTS

MRI Probe with Obliquely Magnetized Magnets

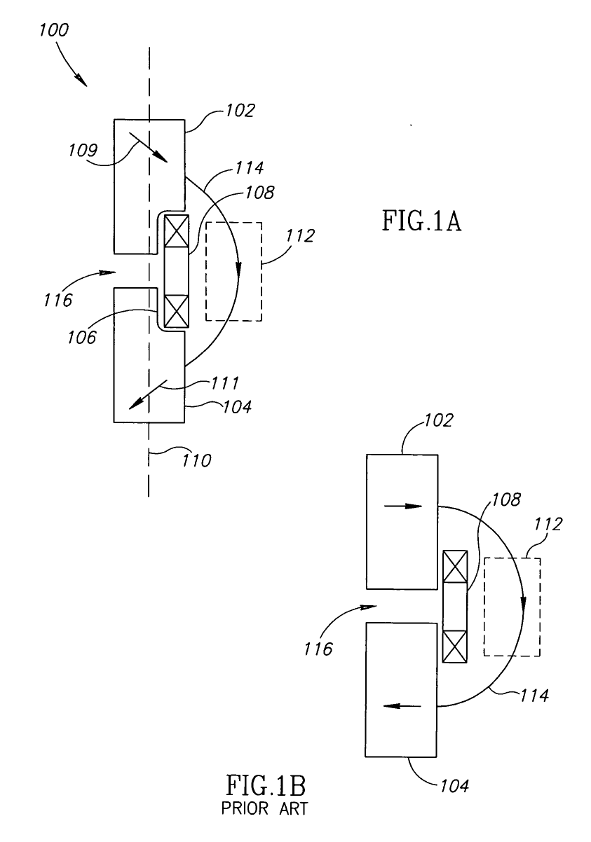

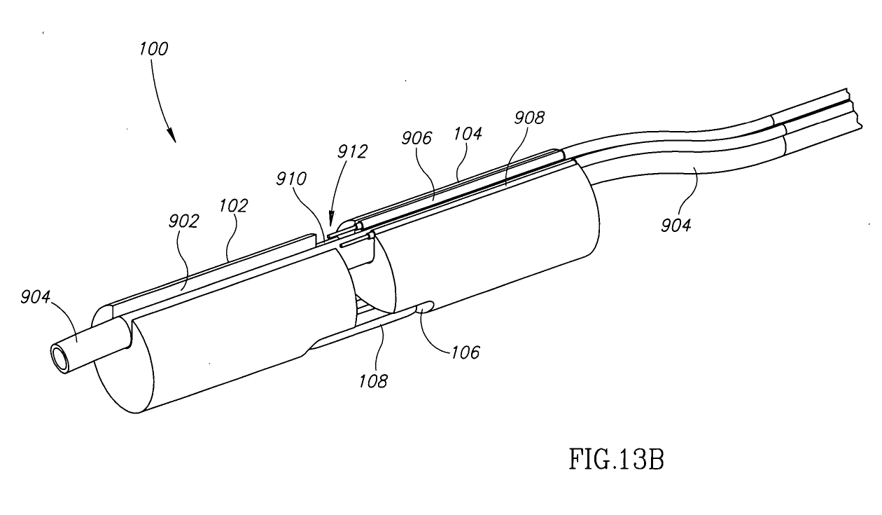

FIG. 1A shows a side-view (x-z plane) cross-section of an MRI probe 100, with magnets 102 and 104. Except for a slot 106, where an RF coil 108 is optionally located, the magnets are circular cylinders, with longitudinal axis (z-axis) 110. RF coil 108, or any of the RF coils shown in the other drawings, is optionally both a transmitting and receiving coil. Alternatively, there are separate transmitting and receiving coils, one or both of them optionally located in slot 106 in the case of probe 100. Optionally, RF coil 108, or an RF coil in any of the other drawings, or one or both of the separate transmitting and receiving coils, is replaced by a different kind of RF antenna. Perspective views of probe 100 are shown in FIGS. 13A and 13B.

This probe is similar to that described by Blank et al, in U.S. Pat. No. 6,704,594, except for the direction of magnetization of the magnets, indicated in FIG. 1A by arrows 109 and 111 on the magnets. Magnet 102 is magnetized at a 45 degree angle pointing down and to the right, while magnet 104 is magnetized at a 45 degree angle pointing down and to the left. In the probe described by Blank et al, on the other hand, shown in a cross-section in the x-z plane in FIG. 1B, a magnet 102 is magnetized to the right, and another magnet 104 is magnetized to the left, with no component of magnetization along the longitudinal axis.

Probe 100 has a field of view 112, bounded approximately in x and z by the box shown in FIG. 1A with angular extent of about 60 degrees. The field of view is the region from which substantial NMR signals are received by RF coil. It is the region in which the static magnetic field produces a nuclear magnetic resonance frequency within the bandwidth of the transmitted RF frequency, and in which the component of the RF magnetic field perpendicular to the static magnetic field is not too small.

In FIG. 1A, magnetic field line 114 extends from magnet 102 to magnet 104, passing through field of view 112, and the static magnetic field is approximately vertical in the field of view. Because both magnets have vertical components which contribute to the magnetic field at the field of view, the magnetic field is greater than it would be at the same distance from the probe if the magnets were magnetized at the same strength purely in a horizontal direction, as in the prior art probe of Blank et al (op. cit.) shown in FIG. 1B. The field is also higher, in field of view 112, in FIG. 1A, than it would be if magnets 102 and 104 were both magnetized in a same vertical direction, as in FIG. 1C, similar to the prior art probes described by Pulyer (op. cite), because the field lines in that case, labeled 114 in FIG. 1C, go mostly across gap 116 between the magnets, and do not extend out to the side very much. The higher magnetic field in the field of view in FIG. 1A results in a higher SNR, or in shorter acquisition time, smaller voxel size, lower RF power, a field of view extending further away from the probe, or a combination of these, to obtain the same SNR.

The magnetic field around probe 100 in FIG. 1A is weaker in either direction away from the x-z plane, and consequently field of view 112 is confined to within a limited range of azimuthal angles. For example, in some embodiments of the invention, the field of view extends about 30 degrees or 45 degrees in each direction from the x-z plane. FIG. 1D shows a cross-section (perpendicular to the longitudinal axis, at the bottom of magnet 102, adjacent to gap 116) of probe 100. Field of view 112 is sector-shaped in this view. The boundaries of field of view 112 shown in FIG. 1D are only representative. The field of view may be defined, for example, as the region within which the received NMR signals have at least half the amplitude for a given proton density, as they have at the location which produces the strongest received NMR signals. The actual field of view depends on the RF bandwidth and on the spatial distribution of the RF field. Alternatively, the field of view may be defined as the region from which an NMR signal with acceptable SNR can be obtained in an acceptable imaging time for in-vivo applications, typically up to 1 minute, in which case the field of view depends also on the RF power and the sensitivity of the antenna. FIG. 1D also shows contours 118 of constant static magnetic field, as solid lines, and contours of constant RF field amplitude 119, as dashed lines.

Optionally, the direction of magnetization of the two magnets is oriented at an angle different from 45 degrees to the longitudinal axis. The optimal angle to maximize the magnetic field about the field of view of the probe, depends on the relative length, diameter and gap distance of the magnets, as well as on where the desired volume of view is located. The optimum angle is optionally determined by calculating the magnetic field about the field of view, for different angles of magnetization of the magnets, using magnetic finite element software. In some cases, the optimal angle may depend not only on maximizing the magnetic field about the field of view, but also on constraints on the magnetic field gradient, which also depends on the angle of magnetization of the magnets. For example, if the RF circuitry is only capable of handling a certain maximum bandwidth, then it may be desired to have a more uniform magnetic field in the field of view, even at the cost of having a lower magnetic field. Or, it may be desirable to have a greater magnetic field at a certain distance from the magnets, to allow the field of view to extend out that far, even at the cost of having less than the greatest possible magnetic field close to the magnets.

Optionally, there is no gap 116 between magnets 102 and 104, but the magnets are bonded together. However, there is a potential advantage to having a gap between the magnets, particularly right behind RF coil 108, where the RF field is greatest. A strong RF field can produce significant eddy currents in the magnets, as well generating sound waves in the magnets at the RF frequency, a phenomenon called magnetoacoustic ringing. Eddy currents and sound waves can both cause heating of the magnets, possibly raising their temperature too high, and wasting RF power, reducing the RF power available for generating MRI signals. Even if the magnets are almost touching each other, having at least a small gap between them, so the magnets are not in electrical contact with each other, or not in mechanical contact with each other, may reduce eddy currents and magnetoacoustic ringing.

Methods of Operation of MRI Probes

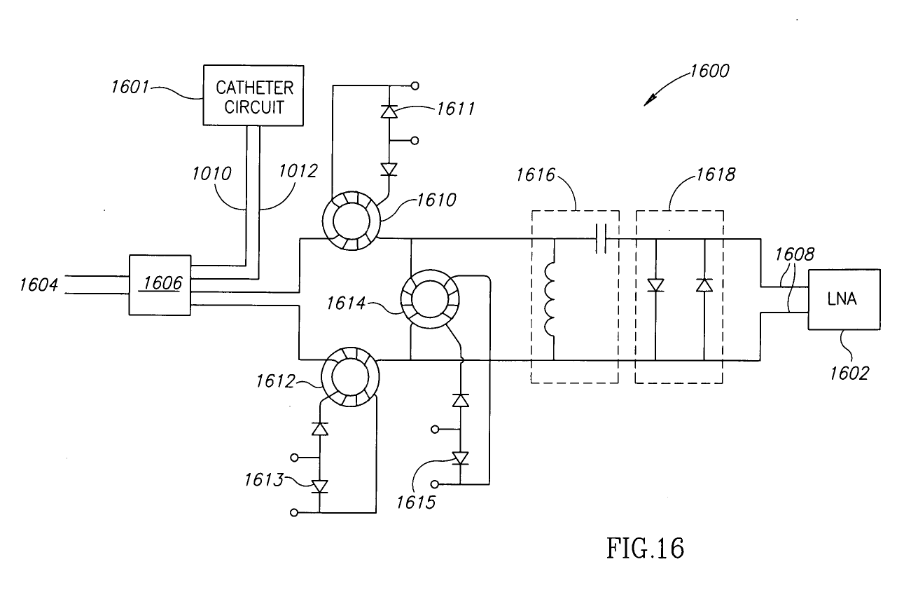

The method of operation of probe 100 is briefly described here with reference to FIG. 1E, which shows MRI system 120. A controller 122, for example a PC, controls an RF power supply 124 through a control cable 126, and controls an interface 128 through a control cable 130. Interface 128 includes elements known in the art such as impedance matching or tuning circuits for the RF coil, amplifiers and filters for the received NMR signals, and switching circuits if the same RF coil is used for transmitting and receiving. One possible embodiment of such a switching circuit is shown in FIG. 16 and described below. The various elements included in interface 128 need not be packaged together in the same physical location, and this is also true of controller 122. Optionally, some or all of the elements of interface 128 are packaged together with some or all of the elements of controller 122, and/or with RF power supply 124.

When told to do so by controller 122, power supply 124 transmits RF power through transmission channel 132 to interface 128. Optionally, the RF power is in the form of pulse sequences known in the art, for example a CPMG spin echo sequence. A catheter 134 carries transmitted RF power from interface 128 to probe 100, which was previously placed in blood vessel 136 using techniques known in the art, or described herein or in the related application filed concurrently, and the RF power excites nuclei in field of view 112, shown in the wall of blood vessel 136. Alternatively, probe 100 is placed in other body cavities, or used outside the body. The excited nuclei emit NMR signals, which are received by probe 100 and carried by catheter 134 from probe 100 to interface 128.

Preferably, for safety reasons, there is an over-power protection circuit, located for example in controller 122 or interface 128, which limits the RF power, averaged over an appropriate time interval, which can be delivered to probe 100. If the average RF power is too high, then the power supply is turned off, and/or the power supply is disconnected from the catheter, and/or other measures are taken to stop the further transmission of RF power to the probe, and optionally an alarm sounds.

Optionally, for safety reasons, interface 128 uses optical coupling to transmit control signals from controller 122 to catheter 134, or to any part of interface 128 that is in electrical contact with catheter 134, and to transmit data signals from catheter 134 to controller 122.

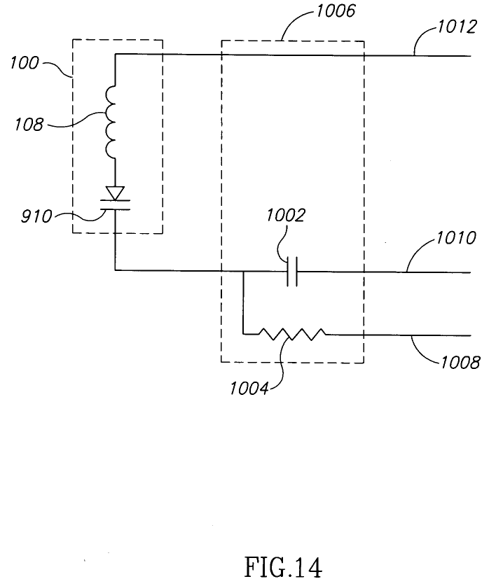

Probe 100 includes magnets and an RF coil for transmitting and receiving, as shown in FIG. 1A, and optionally includes electronic elements that are advantageous to locate in the probe rather in the interface, for example a capacitor and/or a resistor to make the RF coil resonant at the desired range of frequencies, as shown in FIGS. 13A, 13B and 14. Optionally, the electronic elements include elements that are tuned or otherwise controlled by signals sent from interface 128 through catheter 134, and/or elements that send data, other than NMR signals, from the probe to interface 128 through catheter 134.

The NMR signals received by interface 128 are conveyed, optionally after being amplified and/or otherwise processed by interface 128, through receiving channel 138 to controller 122, where the data is optionally used to reconstruct an image of the blood vessel wall, showing, for example, the presence or absence of plaque. Additionally or alternatively, the data is used for other purposes, for example for NMR spectroscopy, or to determine an average T₁, T₂, and/or diffusion rate for the blood vessel wall in the vicinity of the probe. Optionally, the reconstructed image is displayed in real time on a monitor 140. Optionally, the displayed image, or spatially averaged non-imaging information, combines data received at different times from different fields of view as the probe is turned or moved. The MRI system and method outlined in FIG. 1E and described above is also optionally used for the probes shown in the other drawings.

Probe 100 optionally uses one or more of several methods to obtain resolution in the radial, azimuthal and longitudinal directions, in producing an image. The static magnetic field falls off with increasing radial distance from the probe and also with increasing azimuthal angle away from the x-axis, as shown by contours 118 in FIG. 1D. Hence, nuclei are excited only over a limited range of azimuthal angles, for example the range corresponding to field of view 112 in FIG. 1D, which provides azimuthal resolution. Additionally or alternatively, azimuthal resolution is provided by a set of one or more angular gradient coils (not shown), which produce a phase encoding gradient in the azimuthal direction. Radially resolution is optionally provided, within the excited region, by Fourier analysis of each spin echo, treating the radial gradient of the static magnetic field as if it were a read gradient in conventional MRI. Additionally or alternatively, radial resolution is provided by sequentially exciting different radial regions, using different RF frequencies, a method called slice selection in conventional MRI. Each “slice” is bounded approximately by a pair of contours 118 in FIG. 1D. Longitudinal resolution is provided by the finite longitudinal extent of the field of view 112, and optionally by moving the probe longitudinally to a new position and receiving additional NMR signals from the new position.

Optionally, probe 100, or any of the other MRI probes shown in the drawings, uses a high RF bandwidth, and a large number (up to thousands) of spin echoes, in order to obtain a reasonably high SNR with a high field gradient. Alternatively, the probes have an external region of low field gradient, and a more conventional MRI pulse sequence is used, with smaller RF bandwidth and fewer echoes. A potential advantage of using high field gradient in the imaging region, as discussed above, is that the field can be higher in the imaging region, and the imaging region can be broader.

For example, for intravascular MRI probes designed to be used in blood vessels as small as 2 mm in diameter, the field gradient is optionally as great as 50 tesla/meter, or 100 tesla/meter, or 150 tesla/meter, or 200 tesla/meter, or 300 tesla/meter, or 400 tesla/meter, or even higher. The bandwidth is optionally as great as 0.25 MHz, or 0.5 MHz, or 0.75 MHz, or 1 MHz, or 1.25 MHz, or 1.5 MHz, or 2 MHz, or even greater. Note that 1 MHz bandwidth corresponds to 0.024 tesla, so, for example, if the bandwidth is 1 MHz and the gradient is 200 tesla/meter, then the slice has a radial thickness of 0.12 mm. With different values of field gradient and bandwidth, the slice radial thickness is, for example, 0.03 mm, or 0.06 mm, or 0.2 mm, or 0.3 mm, or greater or less than these values.

Another potential advantage of using a large field gradient is that it is easier to measure the diffusion rate of molecules in the tissue being imaged, which can be used to distinguish healthy tissue from the necrotic lipid-rich core in vulnerable plaque. A higher diffusion rate, in the presence of sufficiently strong field gradient, causes the tissue to appear as though it has a shorter effective T₂ value, which depends on the diffusion rate. This effective T₂ value, called Tc, may be more useful that the actual T₂ value for distinguishing vulnerable plaque from healthy vascular tissue. For example, T₂ is typically 30 to 50 milliseconds in vascular tissue, and diffusion rates range from 1.6×10⁻⁹ m²/sec in healthy tissue to 0.4×10⁻⁹ m²/sec in the necrotic core. To have T_{c} depend primarily on the diffusion coefficient rather than on T₂, for this range of diffusion coefficients and T₂, and for a bandwidth of about 1 MHz, the field gradient should be greater than about 100 T/m. Although diffusion of this magnitude can also be measured with smaller field gradients and smaller bandwidth, using fewer echoes of longer duration, the duration and time between echoes should preferably not be much greater than T₂, and in conventional MRI there is a limit to how strong a field gradient can be used, since the gradient is supplied by gradient coils, rather than by the main magnet. Consequently, it is difficult to measure diffusion rates much smaller than 10⁻⁹ m²/sec in conventional MRI. With a self-contained intravascular MRI probe with high static magnetic field gradient and using high bandwidth RF pulses, it is quite possible to measure diffusion rates that are smaller than this by an order of magnitude or more.

MRI Probe with Multiple Sub-Probes

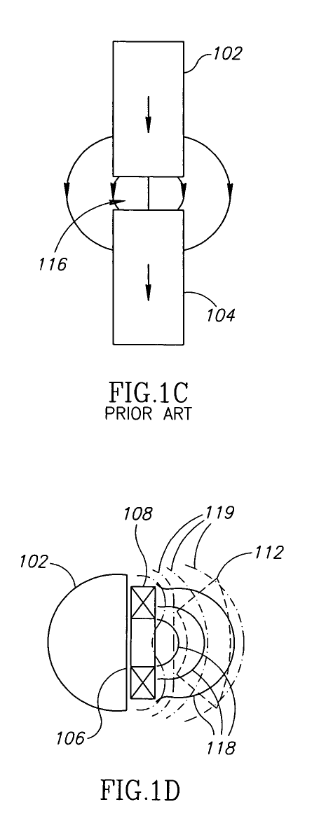

FIG. 2A shows four magnets 202, 204, 206 and 208, arranged in space as they would be along the longitudinal axis of a probe 200. Alternatively, there are more than four magnets, or fewer than four magnets. Each magnet is magnetized in a direction perpendicular to the longitudinal axis, but, as indicated by arrows on the tops of the magnets, the direction of magnetization differs by 90 degrees between one magnet and the next one below it. Probe 200 also has RF coils 210, 212, 214 and 216, each coil associated with one of the magnets, and a structure holding the magnets and coils, not shown in FIG. 2A. Each magnet with its associated RF coil is an independent MRI sensor, which obtains imaging data for its own field of view. Although FIG. 2A shows the coils nearly the same length as the magnets, optionally one or more of the magnets are longer than their associated coils, so that the static magnetic field is more uniform as a function of the longitudinal position in the vicinity of that coil. Optionally the sensors in probe 200, and in all the probes shown in the drawings, are also encapsulated in a biocompatible outer layer, and there are associated electronic components and a catheter, not shown in the drawings.

For each sensor, the RF coil is located against one side of the magnet, centered halfway between the north pole side and the south pole side of the magnet, so the azimuthal locations of the coils also differ by 90 degrees between one sensor and the next one below it. Alternatively the coils are located at a different location against the side of each magnet. However, centering the RF coil halfway between the north pole and south pole of each magnet maximizes the NMR signal, which depends on the component of the RF magnetic field that is perpendicular to the static magnetic field. FIG. 2B shows a cross-sectional view of magnet 202 and RF coil 210 in FIG. 2A, and the magnetic fields they produce, in a plane perpendicular to the longitudinal axis. The fields would look the same for any of the other magnets in FIG. 2A. Magnet 202 produces a static magnetic described by solid field lines 218, which are approximately perpendicular to the RF field, represented by dashed field lines 220. Hence, the probe makes efficient use of the static and RF magnetic fields.

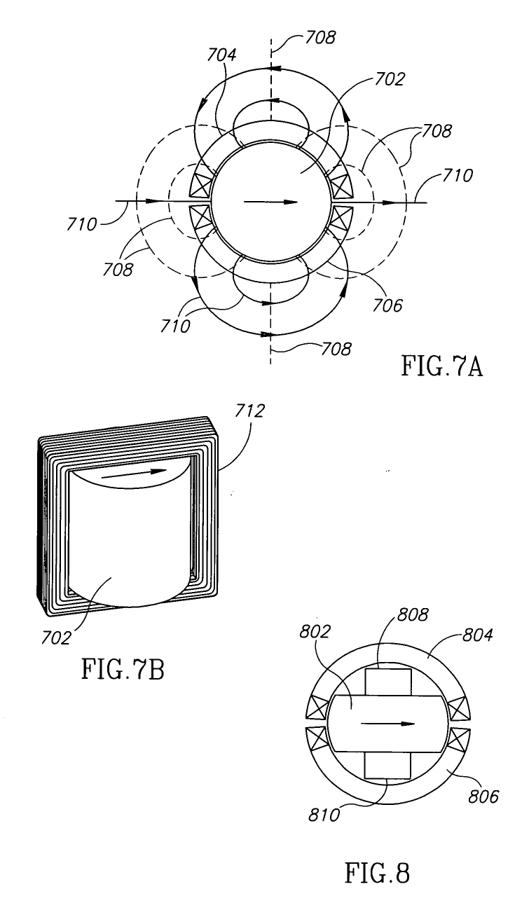

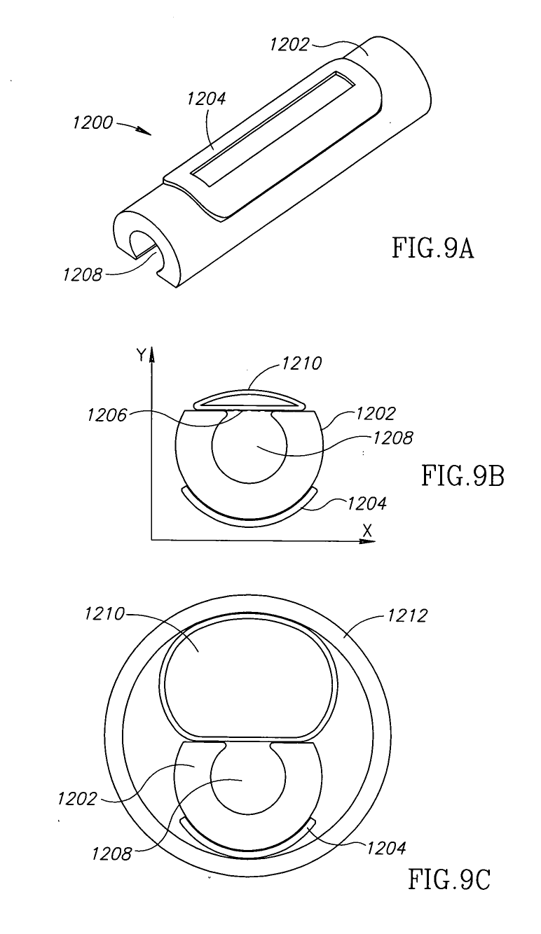

An alternative to the RF coil configuration in FIG. 2A is described below, in FIGS. 7 and 8.

Alternatively, one or more of the sensors in FIG. 2A is replaced by a configuration like probe 100 in FIG. 1A, or like any prior art MRI or NMR probe, or any of the configurations described in the related application being filed concurrently, oriented so that its field of view is inside the wall when the configuration is pressed against the wall. Different sensor designs will generally have fields of view with different azimuthal ranges, and may be advantageous to use in different situations. For example, if the sensor is a large fraction of the diameter of the blood vessel, it may be desirable to have a relatively small field of view azimuthally, in order to obtain good azimuthal resolution, while if the sensor is smaller relative to the diameter of the blood vessel, it may be desirable to have a large field of view, in order to obtain good imaging coverage of the blood vessel wall, without missing anything.

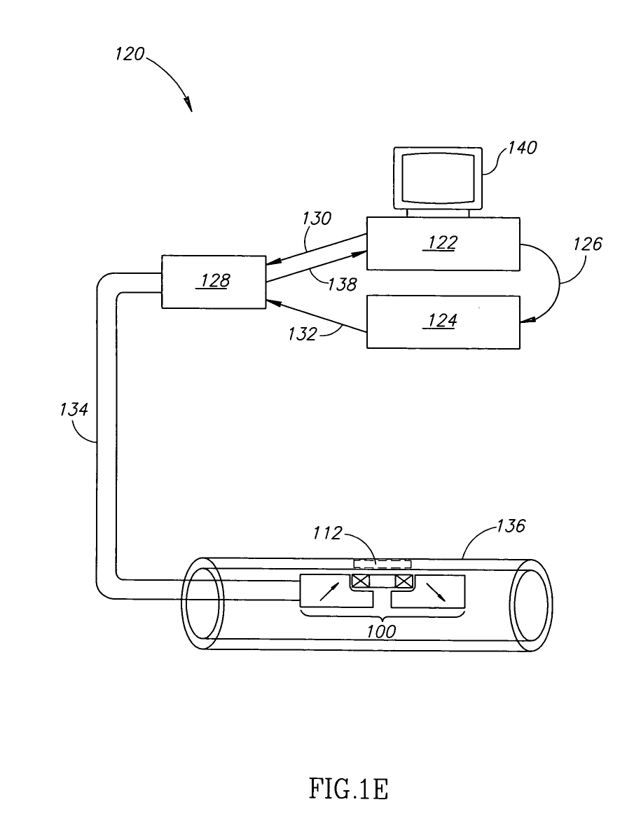

FIG. 3 shows the four sensors of the same probe 200, inside a blood vessel 302. An expandable basket structure 304 is used to move each magnet against the wall of the blood vessel, in the direction where the field of view is centered. Optionally, probe 200 is inserted into and through blood vessel 302 when basket structure 304 is in a retracted state, so that the magnets are lined up, as in FIG. 2A. Once probe 200 has reached a location in the blood vessel where it is desired to make MRI images, for example to look for plaque in the blood vessel wall, then basket structure 304 is expanded. The expansion of basket structure 304 optionally serves two purposes: 1) to hold the magnets and their associated RF coils in place so that images can be made without image quality being affected by relative motion between the blood vessel wall and the magnets, and 2) to bring each magnet close enough to the blood vessel wall so that its field of view extends into the blood vessel wall. Optionally, other devices are used to achieve one or both of these purposes, for example a balloon is used. However, a potential advantage of the basket structure is that it does not occlude the flow of blood.

Preferably, basket structure 304, or whatever expansion mechanism is used, does not press so hard against the blood vessel wall that it might damage plaque, which can be dangerous. For example, the expansion mechanism does not exert a pressure of more than one atmosphere on the blood vessel wall. The field of view for each sensor in probe 200 has a limited azimuthal extent, centered around the RF coil. For example, for each sensor, the field of view is 75 degrees, measured from the center of that sensor. If, as in FIG. 3, the sensors are pressed against the walls of the blood vessel, rather than located in the center of the blood vessel, then the angular range of the field of view measured from the center of the blood vessel will be less than the angular range measured from the center of the sensor. For example, if the field of view is 75 degrees measured from the center of the sensor, it might be 45 degrees measured from the center of the blood vessel. Because the field of view is centered in a different direction for each sensor, and the directions differ by 90 degrees between one sensor and the next, the fields of view for the four sensors together cover half of a full 360 degrees in azimuth, with gaps in coverage no greater than 45 degrees. This may be sufficient to detect any plaque that is large enough to extend more than 45 degrees around the blood vessel wall, and more than the length of the probe along the blood vessel longitudinally, at the location of the probe. Alternatively, the field of view of each sensor is greater than 45 degrees measured from the center of the blood vessel, or there are more than four sensors, and consequently the gaps in imaging coverage are smaller than 45 degrees, or the coverage is a full 360 degrees and there are no gaps in coverage. Optionally, there is even some overlap in the fields of view of the different sensors, and optionally the overlap is used to improve the SNR for the overlapping region, or to obtain information about the dependence of the plaque distribution on the longitudinal coordinate.

Optionally, basket structure 304 is made of a shape memory alloy such as NiTi, and it is expanded by heating it above its transition temperature. Alternatively or additionally, it is made of superelastic shape memory alloy, or it is made of a material other than shape memory alloy, and mechanical means are used to expand the basket structure. For example the two ends of the structure are pulled toward each other by a wire that runs through a catheter, causing the basket structure to bow outward. Alternatively or additionally, a balloon is used to expand the basket structure, although in that case the basket structure may not have the potential advantage of not occluding blood flow.

An expanding structure suitable to use in place of basket structure 304 is described in more detail in the related patent application filed concurrently.

Although the field of view for each sensor in FIG. 3 is centered at a different longitudinal location, in some cases this does not matter very much. For example, plaque in blood vessel walls tends to extend longitudinally for a greater distance than it extends azimuthally and radially, so the azimuthal and radial distribution of plaque in the blood vessel wall may be nearly the same throughout the length of the probe. This may be true, for example, if the probe is less than 15 mm long, since plaque may extend longitudinally over about 15 mm in blood vessels of interest. In that case, if all the sensors together cover a full 360 degrees in azimuth, then the probe may provide a complete map of the distribution of plaque in the vicinity of the probe. A potential advantage of this probe with multiple sensors, over a probe with a single sensor such as that shown in FIG. 1A, for example, is the ability to image a full 360 degrees, or a large fraction of 360 degrees, simultaneously, without the need to rotate the probe.

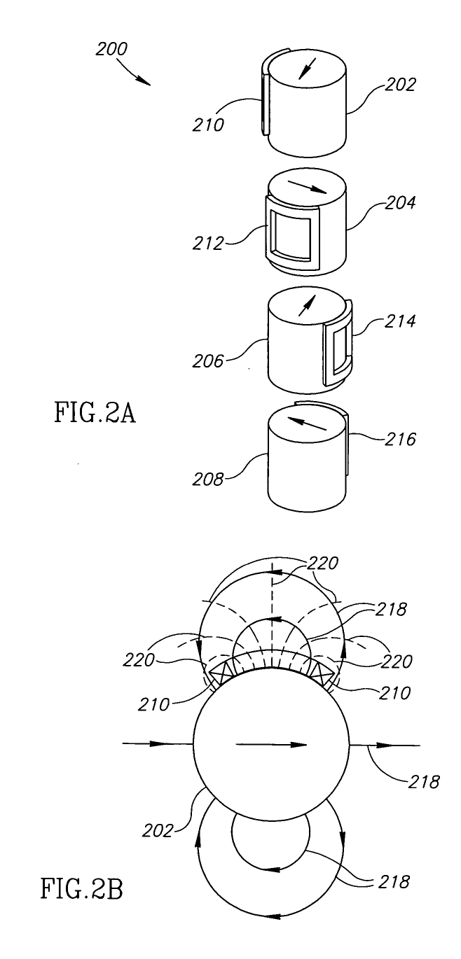

FIG. 4 shows a probe 400, similar to probe 200 in FIG. 3, but with a helical structure 404 holding the sensors against blood vessel wall 302, instead of the basket structure in FIG. 3. Optionally the helical structure, like the basket structure, is in a retracted state when the probe is moved through the blood vessel, and expanded when the probe is in position for imaging. As with the basket structure of FIG. 3, the helical structure of FIG. 4 is optionally made of shape memory alloy, either superelastic or not, and/or various mechanical means known in the art are used to make it expand and retract. Optionally, the helical structure is extended longitudinally to include additional sensors, with fields of view covering the same range of azimuthal angles as magnets 202, 204, 206 and 208, but at a different longitudinal location. For example, the helical structure is longer than 5 mm, or longer than 15 mm, or longer than 30 mm. This allows a range of longitudinal locations on the blood vessel wall to be imaged simultaneously, without the need to move the probe longitudinally. A potential advantage of the helical structure over the basket structure is that the helical structure may be easier to extend longitudinally to an arbitrary length, without changing its basic design. A potential advantage of the basket structure over the helical structure is that the basket structure may be more rugged.

MRI Probes with Longitudinally Magnetized Magnets

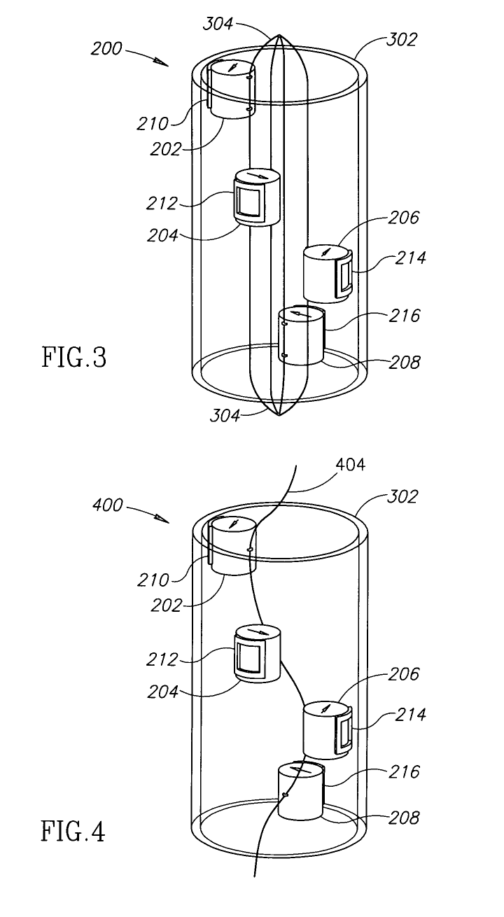

FIG. 5 shows a side view cross-section of a probe 500, with three cylindrical magnets 502, 504 and 506, and an RF coil 508 to one side of the magnets. A field of view 510 is shown as a rectangular region bounded by dashed lines. The magnets are all magnetized longitudinally, but adjacent magnets are magnetized in opposing directions. Flux lines 516, drawn as solid lines, show the direction of the static magnetic field, and flux lines 528, drawn as dashed lines, show the direction of the RF magnetic field, at each point in the plane of the drawing. Preferably, the magnets in probe 500 are touching or closely spaced since, when the magnets are spaced apart, the magnetic field has a saddle point, and hence a region of low field gradient, external to the juncture between the magnets. If probe 500 in FIG. 5 uses a wide RF bandwidth and a large number of spin echoes, as discussed above, then a region of low field gradient is undesirable, since such a region will have poor radial resolution. Even in FIG. 5, in which the two magnets are touching, there are saddle point rings 524 and 526 of the magnetic field right on the surface of the magnets, where the magnets are touching, and there are regions of low field gradient in the vicinity of these saddle point rings. However, if the surface of the magnets is separated by some distance from the blood vessel wall, for example because coil 508 is between the magnets and the wall, then the region of low field gradient can be kept away from field of view 510. A potential advantage of the configuration shown in FIG. 5 is that the magnetic field stays relatively high, further out radially, than for some other magnet configurations.

The probe shown in FIG. 5 has a potential advantage in the use it makes of the static and RF magnetic fields, because the static and RF magnetic fields are perpendicular to each other, and relatively large, over a relatively large volume. At point 530, directly to the right of the center of coil 508 and magnet 504, the static magnetic field, shown by solid line 516, is vertical, pointing downward, and the RF field, shown by dashed line 528, is horizontal, perpendicular to the static magnetic field. (It should be understood that “horizontal” and “vertical” refer to the probe shown in the drawing, but the probe need not be oriented in the direction shown.) At point 532, directly to the right of the boundary between magnets 502 and 504, the static magnetic field is horizontal, pointing to the right, and the RF magnetic field is nearly vertical, again perpendicular to the static magnetic field.

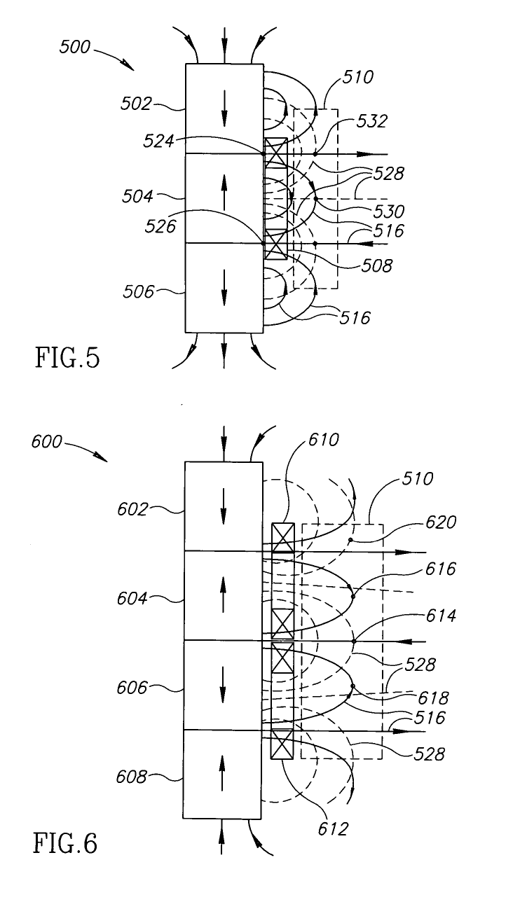

FIG. 6 shows a probe 600, similar to probe 500 shown in FIG. 5, but with four magnets 602, 604, 606 and 608, and two RF coils 610 and 612, instead of three magnets and one coil. As in FIG. 5, the static field is shown by solid lines 516, and the RF field is shown by dashed lines 528. As in probe 500, the magnets in probe 600 are magnetized longitudinally, with abutting magnets magnetized in opposing directions. The two RF coils are run with currents 180 degrees out of phase, which makes the RF magnetic field vertical at point 614, to the right of the center of the probe, where the static magnetic field is horizontal, pointing to the left. At points 616 and 618, the static field is nearly vertical and the RF field is nearly horizontal, while at point 620, the static field is horizontal, pointing to the right, and the RF field is nearly vertical. At intermediate points, the RF and static magnetic fields are also nearly perpendicular to each other.

The larger number of magnets in FIG. 6 produces a field of view 510 which extends a relatively large distance in the longitudinal direction, where the static and RF magnetic fields are fairly high and fairly uniform in magnitude at a given horizontal distance from the probe. In other embodiments of the invention, there are more than four magnets, magnetized alternately in the +z and −z directions as in FIGS. 5 and 6, and with one RF coil adjacent to each magnet except for the magnets at the ends. In some of those embodiments, the field of view extends even further in the longitudinal direction than field of view 510 does in FIGS. 5 and 6.

Optionally, in probe 600 or in a similar probe with more magnets and RF coils, images or other data obtained from the NMR signals received by the different coils are simply averaged together. Alternatively, the NMR signals received by different coils, or by different sets of coils, are not combined together, but are recorded separately and analyzed to obtain finer longitudinal resolution of the field of view.

MRI Probes With Other Magnet Configurations

FIG. 7A shows a cross-section of a magnet 702, normal to the longitudinal axis and passing through the center of the magnet, which could be used for one of the magnets 202, 204, 206 and 208 shown in FIGS. 2A, 3 and 4, but which has an alternative RF coil configuration. Instead of only a single RF coil adjacent to one side of the magnet, centered between the north pole side and south pole side, there are two coils 704 and 706, each extending nearly halfway around the magnet, each coil centered halfway between the north pole and south pole side of the magnet, but on opposite sides of the magnet. The two coils are run in phase, that is to say the currents run in the same direction in the parts of the coil that are adjacent to each other, so that the RF fields from the two coils reinforce each other. The RF magnetic field, indicated by dashed flux lines 708, and the static field, indicated by solid flux lines 710, are both approximately dipoles, perpendicular to each other, and the RF magnetic field and static magnetic field are nearly perpendicular to each other at each point in space. Because the static magnetic field is greater near the poles of the magnet than between the poles, the NMR signal is also greater from this direction, shown to the sides of magnet 702 in FIG. 7A. The smaller the azimuthal extent of the longitudinal portions of the RF coils (the portions where the current is flowing longitudinally), the larger the RF field will be near the poles of magnet 702, further enhancing the directional sensitivity of the probe.

Alternatively, instead of the longitudinal portions of the RF coils being connected by conductors that go around the circumference of magnet 702 azimuthally, as shown in FIG. 7A, the longitudinal portions are connected by conductors that go over the ends of magnet 702. In this case, as shown in FIG. 7B, the two coils are optionally replaced by a single coil 712 which is wound symmetrically around magnet 702, up one side, across the longitudinal axis, and down the other side. All of these coil configurations produce RF magnetic fields similar to those produced by the coil configuration shown in FIG. 7A

FIG. 8 shows a cross-section of a probe, normal to the longitudinal axis, with a magnet 802 and coils 804 and 806, similar to the probe shown in FIG. 7A, and with static and RF field lines similar to those shown in FIG. 7A. Magnet 802 is a circular cylinder, like magnet 702 in FIG. 7A, but with slices taken out of the two sides of the circle, and magnetized in a direction parallel to the flat surfaces where the cylinder was sliced. Optionally, the planes of the slices are not exactly parallel to the axis of the cylinder, but are nearly parallel to the axis, for example within 20 degrees of parallel. Optionally, only one slice is taken out. The space where the slices were taken out is optionally used for electronics packages 808 and 810. Alternatively, only one of the electronics packages is present. If the field of view of the probe is concentrated on the north pole and south pole sides of the magnet, then the parts of the magnet removed by the slices contribute very little to the magnetic field in the field of view, so it is potentially a good use of space to remove this part of the magnet and use it for electronics. Magnet 802 need not be manufactured by starting with a circular cylinder and slicing off one or two pieces, but is described as “a circular cylinder . . . with slices taken out” in order to specify its shape. Use of similar language elsewhere in this application also does not imply that the magnet is necessarily manufactured by starting with a circular cylinder and slicing off one or more pieces, but is intended only to specify a shape.

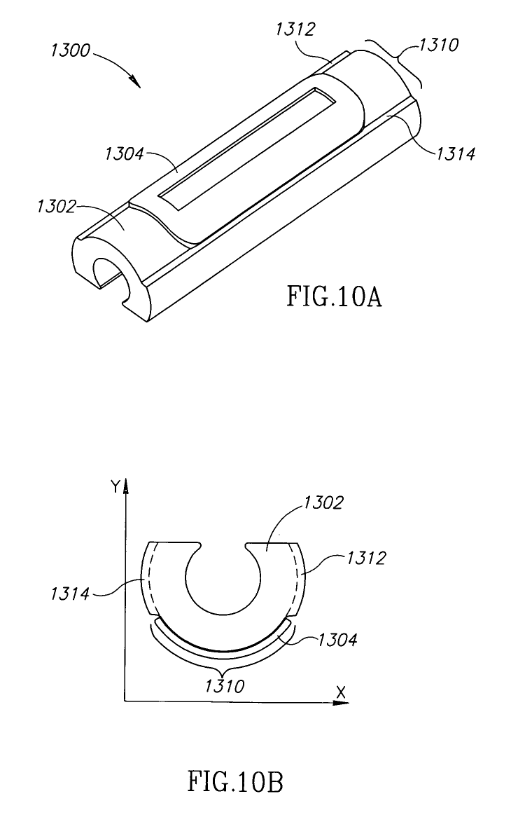

FIG. 9A is a perspective view of a permanent magnet 1202 and an RF coil 1204, for an MRI probe 1200. Probe 1200 could be a stand-alone probe, but it could also be used for one of the sub-probes shown in FIG. 2, FIG. 3, or FIG. 4, for example. FIG. 9B shows a cross-section, perpendicular to the longitudinal axis, of the same probe 1200. Magnet 1202, which is for example 5.3 mm long and 1.6 mm in diameter, is uniformly magnetized in the x-direction, perpendicular to the longitudinal axis and parallel to the chord 1206 where part of the circular cross-section of the magnet has been removed. (Alternatively, the magnet is magnetized in a different direction, but the direction of magnetization shown, together with the location and configuration of RF coil 1204, makes efficient use of the magnet for producing a magnetic field in the imaging region.) The volume of the missing part of the magnet is optionally used for a balloon 1210, shown in a deflated state. FIG. 9C shows balloon 1210 in an expanded state, pressing probe 1200 against a wall 1212 of a blood vessel. Because the missing part of the magnet is opposite the coil, which is adjacent to the imaging region, removing the missing part of the magnet does not significantly reduce the magnetic field in the imaging region. A hollow groove 1208 running along the magnet is optionally used, for example, to allow blood to flow through the probe, or for a guide wire, or for cables to the RF coil, or for more than one of these uses.

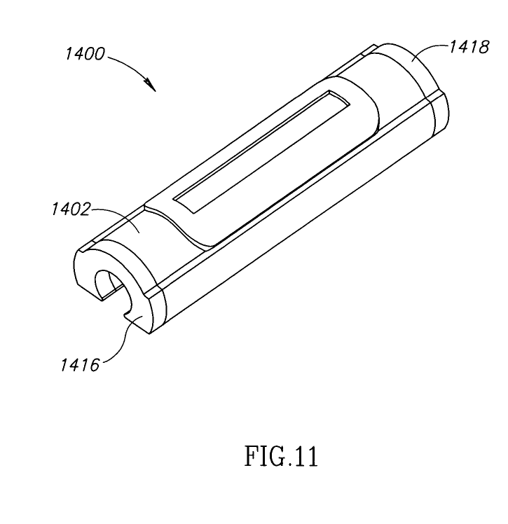

FIG. 10A shows a perspective view, and FIG. 10B shows a cross-sectional view, of another design for a magnet 1302 and RF coil 1304 in a probe 1300. In this design, the RF coil fits into a hollowed out slot 1310 in the surface of magnet 1302. The “ears” 1312 and 1314 of the magnet, which stick out beyond the radius of the coil, provide additional magnetic field strength in the imaging region, compared to probe 1200, but with the same outer envelope. Hence probe 1300 can produce higher signal to noise ratio than probe 1200, for the same RF power and acquisition time.

MRI Probes with End Caps on Magnets

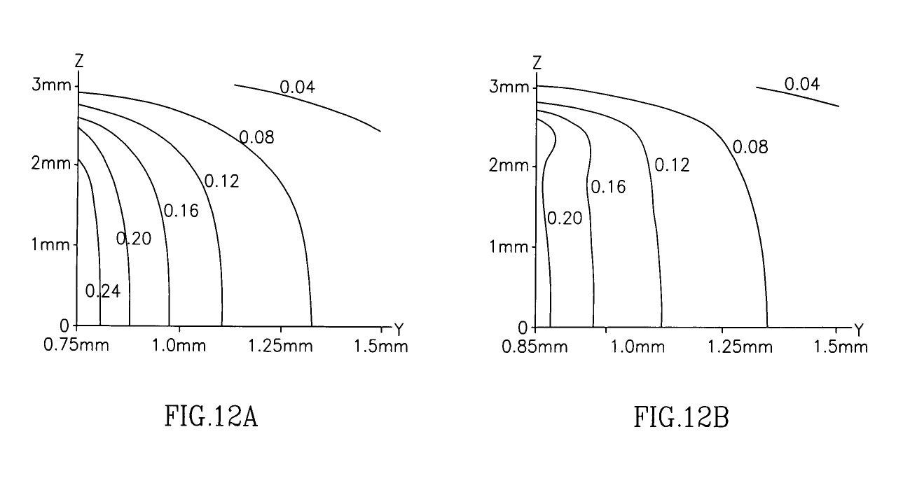

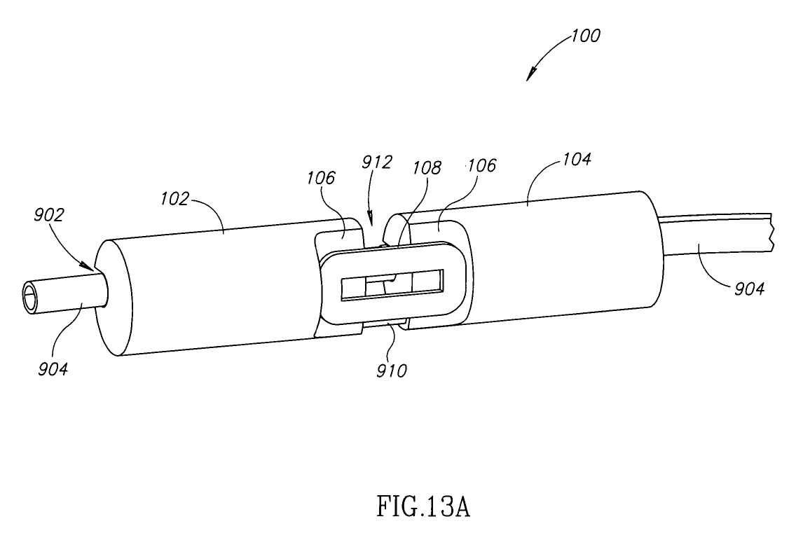

FIG. 11 shows a perspective view of another probe 1400, similar to probe 1300, but with end caps 1416 and 1418 at the ends of a magnet 1402. The end caps need not have the shape and size shown in FIG. 11. Criteria for designing the end caps are given below, in the description of FIGS. 12A and 12B. The cross-section of probe 1400 is the same as the cross-section of probe 1300 shown in FIG. 10B. The end caps, made of a high permeability material such as iron, make the magnetic field more uniform as a function of longitudinal position over the length of the magnet almost out to the ends, but make the field decrease more abruptly near the ends of the magnet.

A probe such as probe 1200 or probe 1300, with magnet magnetized perpendicular to the longitudinal axis, would, if it were infinitely long, produce a magnetic field that is perpendicular to the longitudinal axis everywhere, and independent of axial position. But, with a probe of finite length, the flux lines near the end of the magnet will tend to bow out axially, reducing the field strength, at a given radius and azimuth, near the ends of the magnet. This effect may be significant within one or two magnet diameters away longitudinally from the ends. FIG. 12A shows contours of equal field strength, in the y-z plane passing through the longitudinal axis, for the probe shown in FIG. 10A, on the side of the magnet where the coil and the imaging region are located. The y and z coordinates, which are not to the same scale, are shown in millimeters on the y and z axes, measured from the longitudinal axis of the magnet at the midplane. The magnet extends from z=−2.65 mm to +2.65 mm, and from y=0.32 mm (the radius of the groove) to 0.61 mm (the outer radius of the magnet). Only the region on one side of the midplane of the magnet is shown, since the contours are symmetric about the midplane. Note that the field is significantly lower, for a given value of y, for z close to the end of the magnet (z=2.65 mm), and even for z as much as a millimeter away from the end of the magnet. For example, for y=1.0 mm, the field strength falls to 90% of its midplane value at z=1.8 mm, which is 0.70 magnet diameters from the end of the magnet, and falls to 75% of its midplane value at z=2.3 mm, which is 0.28 magnet diameters from the end of the magnet, and falls to 60% of its midplane value at z=2.65 mm, level with the end of the magnet.

With end caps of sufficient thickness and sufficiently high saturation flux density, much of the flux originating near the ends of the magnet will go through the end caps, instead of bowing out axially past the ends of the magnet. This results in a magnetic field that is substantially more uniform, as a function of axial position, for a given radius and azimuth over most of the length of the magnet, and which falls off substantially more abruptly near the ends of the magnet. For example, the magnetic field at a distance ⅔ of the magnet radius beyond the outer surface of the magnet varies by less than 10% longitudinally between the center of the magnet and a point ⅘ of the magnet radius from the end of the magnet.

This may be seen in FIG. 12B, which is a plot of the same contours of equal field strength, in the y-z plane, for a probe with end caps, similar to the probe shown in FIG. 11. The end caps are each 0.4 mm thick, and the magnet itself extends from z=−2.25 mm to +2.25 mm, so the whole magnet assembly extends from z=−2.65 mm to +2.65 mm, as in the probe shown in FIG. 10A. The magnet has the same inner and outer radius (0.32 mm and 0.61 mm) as in the probe shown in FIG. 10A, and the end caps have a radius of 0.75 mm. Now, at y=1.0 mm, the field strength falls to 90% of its midplane value only at z=2.4 mm, which is 0.21 magnet diameters from the end of the magnet, and falls to 75% of its midplane strength at z=2.65 mm, which is level with the end of the magnet.

The minimum thickness at which the end caps are effective depends on the saturation flux density of the end cap material (typically about 2 tesla for iron or steel), and on the remanence of the permanent magnet material (typically about 1.4 tesla for high quality rare earth magnets), and on the diameter of the magnet. For example, the end caps have a thickness at least 5% of the magnet diameter, or at least 10% of the magnet diameter, or at least 20%, or at least 40%. The probe shown in FIG. 11 has end caps whose thickness is 33% of the magnet diameter.

Optionally, instead of using an end cap to achieve a more uniform magnetic field and a more abrupt fall-off at one or both ends of the magnet, a similar result is achieved by making the magnet thicker toward one or both ends. Magnetic finite element software may be used to calculate the shape of the magnet that would produce the desired magnetic field.

The more uniform magnetic field and abrupt fall off at the ends may have potential advantages. Radial resolution in these probes may be achieved by exciting nuclei in the imaging region at different RF frequencies, corresponding to the magnetic resonance frequency at different field strengths. If the contours of constant field strength are at nearly constant radius, as in FIG. 12B, then it can be possible to distinguish signals coming from different radius. If the contours of constant field strength curve inward toward the ends of the magnet, as in FIG. 12A, then signals coming from nuclei excited by a given RF frequency will come from a range of values of radius. This may make it more difficult to detect plaque and to estimate its proximity to the edge of the lumen, which is an important parameter in assessing its vulnerability, since plaque tends to extend over a substantial length of the blood vessel at constant radius. The higher field gradients near the ends of the magnet in FIG. 12A may also result in shorter effective signal acquisition time, due to diffusion. For both these reasons, a probe with end caps tends to produce images with higher signal to noise ratio, for a given RF power and acquisition time, than a similar probe without end caps.

Manufacturing Process

Regardless of whether the probe comprises multiple sub-probes, and regardless of whether there is more than one magnet or more than one coil in the probe or in each sub-probe, and the direction of magnetization of the magnet or magnets, the same basic procedure is optionally used in assembling the probe or sub-probes. An exemplary procedure may be summarized as follows:

1) Assemble “short” probe (or sub-probe).

2) Assemble electric circuit.

3) Assemble full probe (or sub-probe).

4) Assemble proximal part of catheter.

5) Join catheter to probe (or to each sub-probe).

6) Assemble optional expansion mechanism (e.g. balloon, or basket) on probe or sub-probes.

The “short” probe (or sub-probe), in the terminology used by Topspin Medical, Inc., comprises a magnet or magnets, a structure for mounting the probe on a guide wire or basket, one or more coils, and one or more capacitors (often varicap diodes) for RF tuning, typically in series with a coil. The full probe (or sub-probe) comprises a short probe and an electric circuit, connected with coaxial cables, and a structure joining them together.

FIGS. 13A and 13B show an exemplary layout of the components used in assembling the short probe, for probe 100, the probe design shown in FIG. 1A. FIG. 13A and FIG. 13B show the layout as seen from two different directions, so that all of the components may be clearly seen. Similar components with a similar layout, with appropriate modifications, are optionally used in assembling the short probe or sub-probe for any of the other probe designs described above. Magnets 102 and 104 are each cylindrical, but with a groove 902 running along their back (i.e. on the side of the magnets that will be facing away from the field of view of the probe when the probe is assembled). A tube 904, optionally made of polyimide, is bonded to groove 902, which it fits snugly into. Tube 904 is used, for example, to run a guide wire through, for probe 100. For other probe designs involving multiple sub-probes, a tube similar to tube 904 is optionally used to mount the sub-probes on a structure joining them together, for example a basket structure as in FIG. 3.

Once tube 904 is bonded to groove 902, two coaxial cables 906 and 908 are laid through groove 902 on top of tube 904, and bonded in place against tube 904, for example with cyanoacrylate. For a design of probe 100 with a diameter of 5.5 French (1.83 mm), magnets 102 and 104 are 1.6 mm in diameter, and the coaxial cables are 70 micrometers in diameter. Optionally, the coaxial cables have spiral wrap shielding rather than braided shielding, and leads of cables 906 and 908 are easily stripped by untwisting the shielding. The shielding of the two cables is optionally bonded together with conductive epoxy.

The lead of cable 906 is then soldered to a first pad of a variable capacitor 910, for example a varicap diode whose capacitance can be adjusted by applying a DC voltage to it, for RF tuning, and the soldered bond is optionally strengthened by applying cyanoacrylate. Optionally, cyanoacrylate is used immediately after soldering to strengthen one or more of the soldered bonds in the probe. Variable capacitor 910 is then placed in a gap 912 between the two magnets, and UV curable glue is optionally placed between the variable capacitor and the magnets. Alternatively, another kind of glue is used. Using a 40× microscope, the variable capacitor is optionally then maneuvered precisely into position, for example within 10 micrometers of the magnet surface, and a UV light source is used to cure the UV glue. In the case of probe or sub-probe designs with a single magnet, or with no gap between magnets, variable capacitor 910 is optionally placed in a slot 106 on the front of the magnets (i.e. on the same side of the probe as the field of view), where an RF coil 108 is also located. For example, variable capacitor 910 is placed behind coil 108, or to the side of coil 108. Coil 108 is then placed in slot 106, and optionally is precisely positioned using UV curable glue, or another kind of glue, in the same way as variable capacitor 910 is positioned. Having variable capacitor 910 close to the coil has the potential advantage of reducing stray capacitance and stray inductance in the leads connecting them, improving the efficiency of the RF coupling to the field of view of the probe, and assuring that the probe is tuned to an intended frequency range. Once the variable capacitor and coil are in place, the stripped lead of coaxial cable 908 is soldered to one lead of coil 108, and the second pad of variable capacitor 910 is soldered to the other lead of coil 108. Gap 912 between the magnets, and slot 106, are then optionally filled in with epoxy, forming a smooth cylindrical surface continuous with the cylindrical surface of the magnets.

Optionally, the short probe is then covered with a thin film of vapor deposited aluminum, except for the coaxial cables which are masked, since they are already shielded, in order to shield the RF coil from far-field noise, without shielding out the near-field signal from the imaging region, and without interfering with the transmission of RF power to the near-field imaging region. Heat shrink, for example made of PET, is then optionally placed around the probe and shrunk, further protecting it and preventing any relative movement between the different components of the probe.

To manufacture magnets 102 and 104, for the 5.5 French diameter probe shown in FIG. 1A, the bulk magnet material optionally is first magnetized to 25% of the saturation flux density B. Cylinders are then cut out, 1.8±0.1 mm in diameter and between 5 and 25 mm long, with their axis oriented at an angle of 45 degrees to the direction of magnetization. The cylinders are then ground down to a diameter of 1.6 mm, and cut to the desired length. Groove 902 and slot 106 are then cut in each magnet. The slots and grooves are cut in different sides of magnets 102 and 104, relative to the north and south poles, so that when the probe is assembled magnets 102 and 104 will be magnetized at angles of 90 degrees to each other, as shown in FIG. 1A. The magnets are then magnetized fully.

To manufacture coil 108, the coil is optionally first wound between two flat chrome-coated plates, one of which has a hard steel core, optionally using 20 micrometer insulated copper wire with bonding material on the outside. The coil is then optionally heat-treated to melt the bonding material and to bond the turns together. The coil is then optionally dipped in acrylic and baked, and optionally bent to conform to the shape of the cylinder, as shown in FIGS. 9A and 9B. Another layer of acrylic is optionally applied to the coil, and it is tested for breaks and shorts.

Optionally the coil is wound with wire that is thicker or thinner than 20 micrometers in diameter. However, using wire about 20 micrometers or less in diameter has the potential advantage that the wire diameter is less than the skin depth in copper at the RF frequencies of interest, so that the current penetrates well into all of the wire cross-section. Using wire about 20 micrometers or more in diameter has the potential advantage that it is easier to wind the coil than if thinner wire were used. And using wire about 20 micrometers in diameter has the potential advantage that, for a coil of these dimensions, the coil resistance and inductance have convenient values for impedance matching to off-the-shelf RF amplifiers and power supplies. For example, with 20 micrometer diameter wire, the coil DC resistance is about 25 ohms and the inductance is about 18 microhenries. Alternatively, using different diameter wire or different coil dimensions or a different fill factor or a different material for the coil, the resistance is, for example, about 5 ohms, or 10 ohms, or 50 ohms, or 100 ohms, or greater or less than these values. Alternatively, the inductance is about 3 microhenries, or 8 microhenries, or 40 microhenries, or 100 microhenries, or greater or less than these values. The RF field per ampere produced by the RF coil in the imaging region ranges from about 0.05 tesla/amp to 0.15 tesla/amp. Alternatively, the RF field per amp is about 0.01 tesla/amp or 0.03 tesla/amp or 0.30 tesla/amp, or greater or less than these values.

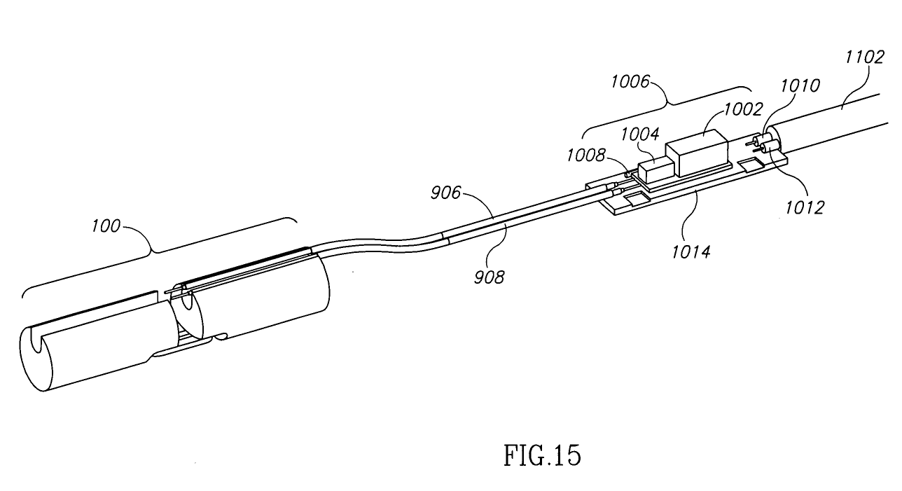

FIG. 14 shows a circuit diagram including RF coil 108 and variable capacitor 910, located in the short probe adjacent to the magnets, and as well as a capacitor 1002 and a resistor 1004, located in an electric circuit 1006 several millimeters away from the short probe. The layout of electric circuit 1006, and its relationship with short probe 100, is shown in FIG. 15. A DC coaxial cable 1008, connected to resistor 1004, is used to apply a DC voltage to variable capacitor 910, to adjust the capacitance of variable capacitor 910, and hence to tune the RF circuit. Capacitor 1002 plus variable capacitor 910 in series comprise the total capacitance of the RF circuit, which, together with the inductance of RF coil 108, determines the tuned frequency of the RF circuit, as seen by AC coaxial cables 1010 and 1012. In addition, the thin film aluminum shielding described above, separated from the RF coil by the thickness of the insulation and the layer of acrylic coating the coil, may act as a stray capacitance, which is preferably taken into account in tuning the circuit.

To assemble electric circuit 1006, as shown in FIG. 15, capacitor 1002 and resistor 1004 are bonded to a printed circuit 1014. AC coaxial cables 1010 and 1012, coming from a catheter 1102, AC coaxial cables 906 and 908, coming from the short probe, and DC coaxial cable 1008, coming from the catheter, are also bonded to printed circuit 1014. The shielding on the leads of the coaxial cables is untwisted and soldered to a ground wire, not shown in FIG. 15, and the coaxial cables are soldered to the appropriate points on printed circuit 1014. The leads of capacitor 1002 and resistor 1004 are also soldered to printed circuit 1014. Cyanoacrylate is optionally applied to the soldered regions, as in the short probe.

Once the short probe and the electric circuit have been assembled, the full probe, comprising the short probe, the electric circuit, coaxial cables 906 and 908 connecting them, guide wire tube 904, and optionally a tube for inflating a balloon attached to the probe, are optionally encapsulated in a tube, for example a polyimide tube, which is filled with epoxy. Optionally, flex molding is optionally bonded to the proximal end of the short probe, and the shaft of the catheter is pulled over the electric circuit, and bonded to the flex molding. Alternatively, the shaft of the catheter is bonded directly to the proximal end of the short probe, but the flex molding may provide greater flexibility.

Optionally, in addition to electric circuit 1006 and the electric elements located in the short probe, there are other electric elements which may be located, for example, in a package at the proximal end of the catheter, outside the body. These other elements may include, for example, one or more amplifiers for amplifying the received RF signal. If the same RF coil is used for receiving and transmitting RF power, then optionally there are elements which isolate the receiving amplifiers from the RF coil when it is in transmitting mode, in order to avoid damaging or saturating the amplifiers. An example of a “duplexer” circuit which incorporates such elements is shown in FIG. 16, and described below. Optionally, the ground wire is a floating ground, for safety reasons. Optionally, the catheter is shielded, to prevent it from acting like an antenna and picking up noise which becomes amplified together with the signal received by the RF coil.

Optionally, the proximal end of the catheter has three branches, for the guide wile, the coaxial cables and ground wire, and the balloon inflating tube, if there is a balloon attached to the probe. The three branches are optionally sealed with epoxy. Optionally, the branch with the guide wire has a special pressure seal, with a side branch for injecting saline solution and heparin, and a swiveler to allow the probe to be rotated. Optionally, the coaxial cables and ground wire are wrapped a few times around the guide wire near the point where the three branches come together, in order to avoid straining the coaxial cables and ground wire when the catheter rotates.

RF Antenna with Short Dead Time

FIG. 16 shows a circuit 1600 which isolates RF coil 108, or any other RF antenna used, from a sensitive Low Noise Amplifier (LNA) 1602, when the RF coil is used as a high power transmitter. When the RF coil is used as a receiver, circuit 1600 connects the RF coil to the LNA, which amplifies the weak received NMR signals. Circuit 1600 is called a “duplexer” because it allows the RF coil to function in a duplex manner, both as a transmitter and a receiver. If the LNA were not isolated from the RF coil during the transmission period, then the LNA would be saturated, and would have a long dead time, much longer than the typical time delay of several microseconds between the transmitted RF pulses and the received NMR signals. The RF power could also damage the LNA if it were not isolated from the RF coil during transmission. Although conventional MRI systems also often use the same RF antenna for transmitting and receiving, the time delay between transmitted RF pulses and received NMR signals is typically milliseconds in conventional MRI, and the RF bandwidth is correspondingly narrow, so a simpler circuit, with a longer dead time and narrower bandwidth, can be used to isolate the receiving amplifier.

During the transmission, high voltage RF pulses coming from transmission line 1604 are coupled to RF coil 108 through a multi-inputs RF transformer 1606. Cables 1010 and 1012, running through the catheter, connect transformer 1606 to RF coil 108, and to the rest of a catheter circuit 1601, shown in more detail in FIG. 14. The transmission pulses are also coupled to line 1608 going to LNA 1602, but with reduced voltage, due to a favorable turn ratio in the transformer. The residual voltage coming out of the transformer to the LNA, during transmission, is attenuated with active toroid protectors 1610, 1612, and 1614. Each toroid protector has primary and secondary turns, where the signal goes through the primary turns and the secondary can either be shorted out or made into an open circuit, by applying a bias voltage of the appropriate sign across PIN diodes 1611, 1613 and 1615, which produces a voltage of the same sign across another pin diode in series with the secondary turns. When the secondary is shorted out, a high current flows through it, magnetically saturating the toroid. The primary of the toroid then shows low RF impedance and the signal passes through easily. The opposite holds when the secondary exhibits an open circuit. Toroid protectors 1610 and 1612 that lie in series to the signal lines exhibit high RF impedance during transmission, while toroid protector 1614 that is in parallel to the lines exhibits RF low impedance during transmission. When the RF coil acts as a receiver, toroid protectors 1610 and 1612 have low RF impedance and toroid protector 1614 has high RF impedance. Following the toroid protectors is a passive high pass filter 1616, to remove any DC or low frequency bias that may be generated during the high voltage pulses. Finally, just before LNA 1602 there are low junction voltage Schottky diodes 1618 that protect the LNA directly and limit the maximum voltage in its input.

Optionally, the resistance of the primary turns of each of the toroid protectors is no more than a few ohms when the secondary circuit is open. Optionally, the apparent resistance of the primary turns is several hundred ohms when the secondary circuit is shorted out. With these values, circuit 1600 will not ring too long (extending the dead time of the LNA) after the RF transmitter is turned off, but the circuit will not dissipate too much power. Optionally, the inductance of the toroid protectors is not more than a few microhenries, which enables good impedance matching at the RF frequencies of interest, on the order of 10 MHz. Optionally, the junction voltage of the Schottky diodes is less than about 0.3 volts, and their capacitance is less than about 1 picofarad, to avoid excessive losses and to provide good coupling to the LNA.

The circuit shown in FIG. 16 is merely representative of the circuits that can be used to isolate the LNA from the RF coil during transmission. As will be understood by one skilled in the art, any of the components shown in FIG. 16 may be configured in different ways. For example, more than two Schottky diodes are used, or the high pass filter comprises more than one capacitor and/or more than one inductor, or it comprises one or more resistors instead of, or in addition to, either the inductor or the capacitor, or additional filters are used in various places in the circuit. Preferably, the circuit includes all of the normal safety features expected in electric circuits used in medical equipment, for example floating grounds, and/or Faraday shields.

The dead time of LNA 1602 is limited by the ringing of circuit 1600, including RF coil 108 and cables 1010 and 1012. A dead time of only 4.5 microseconds has been achieved using the circuit shown in FIG. 16, with a 10 MHz RF frequency. This is as much as an order of magnitude shorter, relative to the RF period, than the shortest dead time achieved previously in similar wide band NMR devices. Optionally, the dead time is about 10 microseconds, or about 2 microseconds, or longer or shorter than these times. The circuit only reduces the SNR of the received signal by 2 dB, relative to an ideal amplifier positioned right near the receiving antenna.

MRI Probe with Non-axisymmetric Balloon

Optionally, probe 100, or any of the other MRI probes shown in the drawings, has a non-axisymmetric elastically expandable balloon mounted on it. The balloon, not shown in the drawings, expands only or mostly on one side of the probe, where the balloon is more elastic, pressing the other side of the probe (where the balloon is more rigid) against the wall of the blood vessel, so that field of view 112 falls inside the wall. The balloon is made, for example, by placing a tube of elastic material around the probe, then masking one side of the tube, and depositing a stiffening material, such as parylene, to the outside of the tube optionally on all sides. When the masking is removed, one side of the tube will be coated with the stiffening material, and will be relatively rigid, while the other side will be free of the stiffening material in the region that was covered by the mask, and will remain elastic. Alternatively, the stiffening material is not deposited on all sides, but only on the side where it is needed, and in this case masking is optionally not used. Optionally, the uncoated part of the balloon is sufficiently thin and elastic so that it can be used in blood vessels with a large range of different diameters, in contrast to the relatively inelastic balloons typically used for expanding stents in arteries.

Thin Film RF Shielding

Optionally, probe 100, or any of the other MRI probes shown in the drawings, is coated with an RF shielding material. Optionally, the RF coil (for example RF coil 108 in probe 100) is coated with the shielding material. The shielding material is much thinner than a skin depth at the RF frequency, but has a conductivity very much greater than the RF frequency times the electrical permittivity of body tissues. The shielding material hardly affects the near-field RF fields that the probe uses to produce MRI images, but largely shields out interference from plane waves at the RF frequency produced by distant sources, much more than a wavelength away. The shielding comprises, for example, a layer of aluminum 300 nanometers thick, applied to the probe by vapor deposition. Optionally, in order to improve the adhesion of the aluminum to the probe or to the RF coil, an even thinner layer of titanium, for example 30 nanometers thick, is applied first.

General

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. A probe, with a longitudinal axis, for use in an NMR system, the probe comprising:
(a) a plurality of static magnetic field sources which create a static magnetic field that is non-axisymmetric about the longitudinal axis, in a region outside the probe; and
(b) at least one antenna, compromising one or more antennas together capable of creating a time-varying magnetic field which is capable of exciting nuclei in a sub-region of the region, and capable of receiving NMR signals from said excited nuclei and generating NMR electrical signals therefrom;
wherein the plurality of magnetic field sources comprise adjacent static magnetic field sources that are magnetized in directions that differ by more than 10 degrees and less than 170 degrees.

2. A probe according to claim 1, wherein said adjacent static magnetic field sources are displaced from each other along the longitudinal axis.

3. A probe according to claim 1, wherein adjacent static magnetic field sources are magnetized in directions that differ by more than 20 degrees and less than 160 degrees.

4. A probe according to claim 1, wherein adjacent static magnetic field sources are magnetized in directions that differ by more than 40 degrees and less than 140 degrees.

5. A probe according to claim 1, adapted for inserting into a cavity in the body.

6. A probe according to claim 5, adapted for inserting into a blood vessel.

7. A probe according to claim 5, adapted for inserting into a blood vessel with inner diameter between 1.5 mm and 6 mm.

8. A probe according to claim 5, adapted for inserting into a blood vessel with inner diameter between 2 mm and 4 mm.

9. A probe according to claim 1, wherein the static magnetic field sources comprise a first magnetic field source and a second magnetic field source, both with longitudinal components of magnetization having a same sign, and with transverse components of magnetization differing in direction by more than 90 degrees.

10. A probe according to claim 9, wherein there is a gap between the first and second magnetic field sources.

11. A probe according to claim 9, wherein the transverse components of magnetization differ in direction by more than 140 degrees.

12. A probe according to claim 11, wherein the transverse components of magnetization differ in direction by more than 160 degrees.

13. A probe according to claim 9, wherein the ratio of the magnitude of the transverse and longitudinal components of magnetization is greater than 0.5 and less than 2, for both the first and second magnetic field sources.

14. A probe according to claim 13, wherein the ratio is between 0.8 and 1.2, for both the first and second magnetic field sources.

15. A probe according to claim 9, wherein at least one of the at least one antennas extends over a range in the longitudinal direction that overlaps the longitudinal ranges of both the first and second magnetic field sources, and is located on one side of the longitudinal axis.

16. A probe according to claim 15, wherein the center of said antenna is located within 60 degrees of the location at which the longitudinal component of the static magnetic field is greatest, for that longitudinal position and distance from the longitudinal axis.

17. A probe according to claim 16, wherein the center of said antenna is located within 30 degrees azimuthally of said location.

18. A probe according to claim 15, wherein the first and second magnetic field sources extend radially to the surface of a smallest convex volume which includes both magnetic field sources, except for a slot carved into one or both of the first and second magnetic field source, and said antenna is located in one or both slots, entirely within said smallest convex volume.

19. A probe according to claim 18, wherein the smallest convex volume is cylindrical.

20. A probe according to claim 1, wherein the static magnetic field sources each have a component of magnetization transverse to the longitudinal axis that has a magnitude more than 2 times the magnitude of the longitudinal component of magnetization.

21. (canceled)

22. A probe according to claim 20, wherein the transverse components of magnetization of adjacent static magnetic field sources differ in direction by more than 40 degrees and less than 140 degrees.

23. A probe according to claim 20, wherein the at least one antennas comprise an antenna associated with each of the static magnetic field sources.

24. A probe according to claim 23, wherein, for each of said antennas, the static magnetic field in the extended sub-region is at least 80% produced by the static magnetic field source which that antenna is associated with.

25. A probe according to claim 24, wherein each sub-region has a limited range of azimuthal angles, and the azimuthal direction of the center of the range differs by more than 40 degrees and less than 140 degrees for at least two antennas associated with adjacent static magnetic field sources.

26.-34. (canceled)

35. A probe according to claim 25, and including an expansion mechanism with a contracted state and an expanded state, which, when it expands, moves at least two of the magnetic field sources, and their associated antennas, in different directions transverse to the longitudinal axis.

36.-38. (canceled)

39. A probe according to claim 35 which is adapted to be inserted into a lumen of inner diameter greater than a minimum size, and wherein, when the imaging probe is inserted into a lumen of inner diameter twice the minimum size and the expansion mechanism is in its expanded state, the at least two static magnetic field sources and their associated antennas are close enough to the wall of the lumen so that at least part of the sub-region of each of their associated antennas is inside the wall.

40. (canceled)

41. A probe according to claim 39, wherein the parts of said sub-regions within the wall cover a set of azimuthal angles around the wall that does not have any gap greater than 90 degrees.

42.-44. (canceled)

45. A probe according to claim 41, wherein the parts of said subregions within the wall cover said set of azimuthal angles within a longitudinal range of less than 15 mm.

46.-51. (canceled)

52. A probe according to claim 20, and including an expansion mechanism with a retracted state and an expanded state, which mechanism, when it expands, moves at least two of the static magnetic field sources in different directions transverse to the longitudinal axis.

53. A probe according to claim 52 which is adapted to be inserted into a lumen of inner diameter greater than a minimum size, and wherein when the imaging probe is inserted into a lumen of inner diameter twice the minimum size and the expansion mechanism is in its expanded state, the probe presses against the wall of the lumen with sufficient force to stabilize the position of the probe sufficiently so that relative motion of the probe and the wall does not substantially affect the image quality.

54.-65. (canceled)

66. A probe according to claim 1, wherein the sub-regions together have a longitudinal extent greater than 20% of the length of the probe in the longitudinal direction.

67. A probe according to claim 1, wherein the sub-regions together have a longitudinal extent greater than 50% of the length of the probe in the longitudinal direction.

68. A probe according to claim 1, wherein the sub-regions together have a longitudinal extent greater than 2 mm.

69. A probe according to claim 68, wherein the sub-regions together have a longitudinal extent greater than 5 mm.

70. A probe according to claim 69, wherein the sub-regions together have a longitudinal extent greater than 15 mm.

71. A probe according to claim 70, wherein the sub-regions together have a longitudinal extent greater than 30 mm.

72. A probe according to claim 1, wherein at least one of the static magnetic field sources is a permanent magnet element in the shape of a cylinder with a piece sliced off, the plane of the slice being within 20 degrees of parallel to the axis of the cylinder, the permanent magnet being magnetized in a direction substantially perpendicular to the axis of the cylinder and parallel to the plane of the slice.

73.-78. (canceled)

79. A probe according to claim 1, wherein the at least one antenna comprises a coil.

80.-82. (canceled)

83. A probe according to claim 1, wherein the plurality of static magnetic field sources comprise a plurality of permanent magnets.

84.-94. (canceled)

95. A probe according to claim 1, wherein the plurality of static magnetic field sources comprise a permanent magnet with substantially uniform cross-section transverse to the longitudinal axis, magnetized substantially uniformly in a direction substantially perpendicular to the longitudinal axis, and including at least one end cap, located at one end of the permanent magnet sufficiently thick and permeable to make the magnetic field at a distance ⅔ of the magnet radius beyond the outer surface of the magnet vary by less than 10% longitudinally between the center of the magnet and a point ⅘ of the magnet radius away from said end of the magnet.

96.-99. (canceled)

100. A probe according to claim 1, wherein the time-varying magnetic field differs in direction from the static magnetic field by more than 60 degrees and less than 120 degrees, somewhere in the sub-region.

101. A probe according to claim 1, wherein at least one of the static magnetic field sources comprises a material with skin depth greater than the largest dimension of said static magnetic field source, at the proton nuclear resonance frequency at the maximum static magnet field in the region outside the probe.

102. A probe according to claim 1, wherein at least one of the static magnetic field sources comprises sintered material.

103. A probe according to claim 1, wherein at least one of the static magnetic field sources comprises ferrite.

104. A probe according to claim 1, wherein the probe is an imaging probe, and the NMR system is an MRI system.

105. A probe according to claim 1, wherein the one or more antennas comprise a single antenna capable of creating the time-varying magnetic field, and receiving the NMR signals and generating the NMR electrical signals.

106. A probe according to claim 1, wherein the one or more antennas comprise:
(a) a transmitting antenna capable of creating the time-varying magnetic field; and
(b) a receiving capable of receiving the NMR signals and generating the NMR electrical signals.

107. An NMR system comprising a probe according to claim 1, a power supply which transmits power to at least one of the antennas of the probe to create the time-varying magnetic field, and a data analyzer which reconstructs NMR characteristics of material in the sub-region from the NMR electrical signals generated by at least one of the antennas of the imaging probe.

108. An NMR system according to claim 107, wherein all of the at least one antennas that the power supply transmits power to are different from all of the at least one antennas that generate the NMR electrical signals from which the data analyzer reconstructs the NMR characteristics.

109. Au NMR system according to claim 107, wherein at least one of the at least one antennas both creates the time-varying magnetic field and generates the NMR electrical signals from which the data analyzer reconstructs the NMR characteristics.

110. An NMR system according to claim 107, wherein the NMR system is an MRI system, the probe is an imaging probe, and the data analyzer comprises an image reconstructor which reconstructs an image.

111. An NMR system comprising:
(a) a self-contained NMR probe with an RF antenna used for transmitting RF pulses and receiving NMR signals;
(b) an amplifier for amplifying the NMR signals; and
(c) an electric circuit, comprising active toroid protectors, which circuit isolates the amplifier from the RF antenna when the RF antenna is transmitting RF pulses, and connects the amplifier to the RF antenna when the RF antenna is receiving NMR signals.

112. A non-imaging NMR system comprising:
(a) a probe, adapted for use inside the body, comprising a static magnetic field source which generates a static magnetic with at least one saddle point in a region outside the probe, and at least one antenna, comprising one or more antennas together capable of creating a time-varying magnetic field which is capable of exciting nuclei in a sub-region of the region, and capable of receiving NMR signals from said excited nuclei and generating NMR electrical signals therefrom;
(b) a power supply which transmits power to at least one of the antennas of the probe to create the time-varying magnetic field; and
(c) a data analyzer which reconstructs NMR characteristics, other than spectroscopic data, of material in the sub-region from the NMR electrical signals generated by at least one of the antennas of the imaging probe, but which data analyzer does not reconstruct images.
